SEQUENCE LISTING (18 sequences)

SEQ ID NO: 1 - protein, 102 aa, Artificial Sequence
  synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 2 - protein, 102 aa, Artificial Sequence
  artificial sequence
  misc_feature (1)..(1): Xaa can be any naturally occurring amino acid
XLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 3 - protein, 102 aa, Artificial Sequence
  artificial construct
  misc_feature (1)..(1): Xaa can be any naturally occurring amino acid
XLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPN

SEQ ID NO: 4 - protein, 108 aa, Artificial Sequence
  artificial construct
  misc_feature (7)..(7): Xaa can be any naturally occurring amino acid
MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPN

SEQ ID NO: 5 - protein, 120 aa, Artificial Sequence
  artificial construct
  misc_feature (7)..(7): Xaa can be any naturally occurring amino acid
MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSHHHHHHC

SEQ ID NO: 6 - protein, 120 aa, Artificial Sequence
  artificial construct
MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCHHHHHH

SEQ ID NO: 7 - protein, 125 aa, Artificial Sequence
  artificial construct
MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCGGGGSHHHHHH

SEQ ID NO: 8 - protein, 130 aa, Artificial Sequence
  artificial construct
MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSGGGGSCGGGGSHHHHHH

SEQ ID NO: 9 - protein, 124 aa, Artificial Sequence
  artificial construct
MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSGGGGSCGGGGS

SEQ ID NO: 10 - protein, 36 aa, Artificial Sequence
  artificial construct
MIKIATRKYLGKQNVYDIGVERDHNFALKNGFIASN

SEQ ID NO: 11 - protein, 36 aa, Artificial Sequence
  artificial construct
MIKIATRKYLGKQNVYGIGVERDHNFALKNGFIASN

SEQ ID NO: 12 - protein, 36 aa, Artificial Sequence
  artificial construct
MIKIATRKYLGKQNVYGIGVERDHNFALKNGFIAHN

SEQ ID NO: 13 - protein, 287 aa, Artificial Sequence
  artificial construct
MIKIATRKYLGKQNVYGIGVERDHNFALKNGFIAHNFFNGTVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLEHHHHHH

SEQ ID NO: 14 - protein, 36 aa, Artificial Sequence
  artificial construct
MIKIATRKYLGKQNVYGIGVERDHNFALKNGFIAHA

SEQ ID NO: 15 - protein, 42 aa, Artificial Sequence
  artificial construct
MHHHHHHIKIATRKYLGKQNVYGIGVERDHNFALKNGFIAHA

SEQ ID NO: 16 - protein, 42 aa, Artificial Sequence
  artificial construct
MIKIATRKYLGKQNVYGIGVERDHNFALKNGFIAHAHHHHHH

SEQ ID NO: 17 - protein, 287 aa, Artificial Sequence
  artificial construct
MIKIATRKYLGKQNVYGIGVERDHNFALKNGFIAHNMFNGTVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLEHHHHHH

SEQ ID NO: 18 - protein, 120 aa, Artificial Sequence
  artificial construct
  misc_feature (7)..(7): Xaa can be any naturally occurring amino acid
MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCHHHHHH

(19) United States
(12) Patent Application Publication
(10) Pub. No.: US 2023/0174574 A1
(43) Pub. Date: Jun. 8, 2023

(54) METHODS AND COMPOSITIONS FOR ENHANCING STABILITY AND SOLUBILITY OF SPLIT-INTEINS

(71) Applicant: Ohio State Innovation Foundation, Columbus, OH (US)

(72) Inventors: David Webster WOOD, Dublin, OH (US); Joseph TARIS, Columbus, OH (US)

(21) Appl. No.: 17/922,472

(22) PCT Filed: Apr. 30, 2021
(86) PCT No.: PCT/US2021/030161

§ 371 Date: Oct. 31, 2022

Related U.S. Application Data
(60) Provisional application No. 63/018,084, filed on Apr. 30, 2020.

(51) Int. Cl. C07K 1/22; C07K 14/47

(52) U.S. Cl. CPC C07K 1/22; C07K 14/47

(57) ABSTRACT

Disclosed herein is a protein purification system and methods of making such a system. Specifically, the invention relates to a method of immobilizing an N-terminal intein segment to a solid support, the method comprising: exposing an N-terminal intein segment to a cognate folding partner under conditions that promote association between the N-terminal intein and the cognate folding partner; immobilizing the N-terminal intein to a solid support; subjecting the N-terminal intein to conditions that disrupt association between the N-terminal intein and the cognate folding partner; and washing the solid support to remove non-bound material, thereby immobilizing an N-terminal intein segment to a solid support.

### CROSS-REFERENCE TO RELATED APPLICATIONS

This application claims benefit of U.S. Provisional Application No. 63/018,084, filed Apr. 30, 2020, incorporated herein by reference in its entirety.

### STATEMENT REGARDING FEDERALLY SPONSORED RESEARCH

This invention was made with government support under grant R21GM126543 awarded by the National Institutes of Health (NIH). The government has certain rights in the invention.

### BACKGROUND

Inteins are naturally occurring, self-splicing protein subdomains that are capable of excising out their own protein subdomain from a larger protein structure while simultaneously joining the two formerly flanking peptide regions (“exteins”) together to form a mature host protein.

The ability of inteins to rearrange flanking peptide bonds, and retain activity when in fusion to proteins other than their native exteins, has led to a number of intein-based biotechnologies. These include various types of protein ligaton and activation applications, as well as protein labeling and tracing applications. Split inteins have recently gained attention for affinity chromatography applications, where an N-Intein Ligand - one distinct protein of a specific pair - is expressed recombinantly in standard cell culture techniques (usually microbial expression) then subsequently immobilized onto a solid chromatography support media (resin, beads, membranes, and the like). The N-Intein Ligand will comprise an N-terminal intein (INT_{N}) segment, which can be modified and additionally may comprise functional groups that aid in purification, immobilization or functional modulation of the INT_{N} segment. To be used for protein purification, a counterpart C-terminal intein segment ‘tag’ is expressed in fusion with a given target protein and is then captured by the immobilized N-Intein Ligand, thereby acting as a self-cleaving affinity tag to facilitate purification of the target protein (e.g., as described in U.S. Pat. #10,066,027 B2). However, in order for self-cleaving tag applications to be enabled, the N-Intein Ligand must be economically manufactured in a recombinant system, purified and immobilized onto a solid substrate.

Effectively, the overall yield in any conventional protein manufacturing process is fundamentally limited by the total amount of protein that is produced in cell culture, and the percentage of that protein which remains soluble when extracted from the cells. Regardless of how efficiently a recombinant protein is produced in cell culture though, only soluble proteins can be recovered and purified by conventional chromatography techniques, meaning any protein forming insoluble aggregates upstream - either during expression, harvest, lysis, clarification or filtration steps - will be lost and discarded in the manufacturing process. In some cases, proteins that are expressed as insoluble aggregates can be recovered and refolded in vitro as part of the purification process, but the required refolding processes are difficult to develop and are typically inefficient.

Standard microbial fermentation techniques are capable of over-expressing recombinant N-Intein Ligands at moderately high expression titers, but due to the inherent structure of the protein - or lack thereof - the resulting protein is prone to aggregation, vulnerable to degradation, and is often insoluble when extracted from its cellular host. This has made it uncommonly difficult to construct a reliable and economically viable process to manufacture the N-Intein Ligands. Indeed, a majority - sometimes upwards of 90% - of the total protein expressed in fermentation appears to be insoluble after cell lysis and is lost during manufacturing. The resulting net yield of soluble N-Intein Ligand from standard E. coli expression is on the order of 10-30 mg protein per liter of expression culture, which is approximately two orders of magnitude lower than most commercially operating recombinant protein manufacturing processes. This directly and proportionally drives the cost of goods and cost of production for split-intein mediated affinity chromatography platforms, and existentially endangers their commercial viability.

In general, solubility is a common issue with heterologous expression that scientists and engineers have been fighting since protein engineering first began - many potential solutions have been employed with various degrees of success. These most commonly focus either on promoting proper structural assembly in vivo, or harsh chemical refolding treatments to resolubilize the aggregate ex vivo. Numerous approaches to promote proper folding of the N-intein have been attempted in vivo, which have shown moderate yet inconsistent improvements to net soluble recovery in manufacturing (e.g., as described in Millipore patent application WO 2016/073228 A1 and GE patent application US 2019/0263856 A1). It appears that even when expressed properly folded and soluble in cell culture, the protein is still highly sensitive to spontaneous idiopathic aggregation at inconsistent and unpredictable amounts, even under identical ex vivo handling conditions. This observation is reinforced by structural studies of the wild-type INT_{N} segments published in the literature by other research groups (Shah, Eryilmaz et al. 2013).

Therefore, what is needed are methods and compositions for heterologous protein expression of split-inteins that greatly increase solubility of the expressed product and stability in downstream manufacturing processes.

### SUMMARY

In accordance with the purpose(s) of the invention, as embodied and broadly described herein, the invention, in one aspect, relates to a method of stabilizing an N-Intein Ligand during expression and purification, purifying the N-Intein Ligand, and immobilizing the N-Intein Ligand to a solid support. In particular, disclosed is a method comprising: forming a soluble and stable intein complex via assembly of the N-Intein Ligand with a Cognate Binding Partner (e.g., a corresponding C-terminal intein segment; alone or in fusion to a cleavable or non-cleavable fusion partner); purifying the intein complex; and immobilizing the intein complex to a solid support. The intein complex can then be subjected to conditions that disrupt association between the N-Intein Ligand and the cognate binding partner; and the solid support washed to remove non-bound Cognate Binding Partner; and conditions provided that allow the N-Intein Ligand to fold into an active state.

The Cognate Binding Partner can comprise a C-terminal intein (INTc) segment that binds an N-Intein Ligand to induce a structured, soluble intein complex. The N-Intein Ligand and the Cognate Binding Partner can be co-expressed either in vivo in a single cell from a single plasmid or two-plasmid system, or in trans (expressed in separate cells) and mixed before or during the purification process. Such immobilization can take place onto a solid support, such as chromatographic media, a membrane, or a magnetic bead. In one example, the chromatographic media can be a solid chromatographic resin backbone.

Utilizing a Cognate Binding Partner to stabilize the N-Intein Ligand renders the N-Intein Ligand incapable of binding any other INTc segment. Therefore, following immobilization, the N-Intein Ligand must be denatured or otherwise dissociated from the Cognate Binding Partner, allowing the Cognate Binding Partner to be removed, washed, or “stripped” away from the N-Intein Ligand. Once the Cognate Binding Partner is removed, the immobilized N-Intein Ligand must be reverted to an active state (capable of binding new partner), thereby forming a functional affinity capture medium.

Disclosed is a method for manufacturing an affinity medium comprising an N-Intein Ligand covalently bound to a convenient substrate, as well as compositions related to the manufacturing process. The N-Intein Ligand can comprise an internal N-terminal intein segment (INT_{N}) along with operably linked fusion partners. The INT_{N} segment within the N-Intein Ligand can been derived from a native intein such as the Npu DnaE intein. The INT_{N} segment may further be modified to increase its utility (e.g., so as to not comprise any cysteine residues within the INT_{N} segment, thus promoting single-point attachment to a substrate). For example, a tag can be attached to the INT_{N} segment within a region following the C-terminal residue of the INT_{N} segment so as to aid in purification, detection, and/or enhancement of soluble expression of the N-Intein Ligand. The N-Intein Ligand can also comprise amino acids within a region following the C-terminal residue of the INT_{N} segment, which allow for covalent immobilization of the N-Intein Ligand onto a substrate. The N-Intein Ligand can further comprise a sensitivity-enhancing motif, which renders its cleaving activity highly sensitive to extrinsic conditions. The sensitivity-enhancing motif can be in fusion to the N-terminus of the INT_{N} segment. The extrinsic condition can be pH, temperature, zinc ion concentration, or a combination of these.

Also disclosed is a protein purification medium, wherein the medium comprises an N-Intein Ligand covalently immobilized on a solid support, wherein 90% or more of the N-Intein Ligand molecules are associated with Cognate Binding Partners, and wherein at least 90% of the cognate binding partners are not expressed in fusion with a desired protein of interest. The Cognate Binding Partner can comprise an INTc segment that binds an N-Intein Ligand to induce a structured, soluble intein complex.

Further disclosed is a protein purification medium, wherein the medium comprises N-Intein Ligand covalently attached to a solid support, and further wherein greater than .001% of the N-Intein Ligand molecules are associated with cognate binding partners, and wherein at least 90% of the cognate binding partners are not expressed in fusion with a desired protein of interest. Again, the Cognate Binding Partner can comprise an INTc segment that binds an N-Intein Ligand to induce a structured, soluble intein complex.

Also disclosed is a chromatographic resin comprising a base resin with covalently-bound N-Intein Ligands, wherein the resin’s measured compressibility differential (ΔC) is less than about 1, 2, 3, 4, 5, 6, 7, 8, 9, or 10%, as compared to its base resin substrate.

Also disclosed is a chromatographic resin comprising a base resin with covalently-bound N-Intein Ligands, wherein the resin’s measured intrinsic functional compressibility factor (IFCF) is between 1.10 and 1.25.

Also disclosed is an expression vector comprising exogenous nucleic acid, wherein the exogenous nucleic acid encodes an N-Intein Ligand and a Cognate Binding Partner, wherein the N-Intein Ligand can be encoded to be expressed with a purification tag, and wherein the Cognate Binding Partner may not be encoded for expression in fusion with a desired protein of interest. Also disclosed is a two-plasmid system wherein the N-Intein Ligand and Cognate Binding Partner are encoded on two distinct compatible plasmids housed within a single cell. Also disclosed is a cell comprising the expression vector(s). The Cognate Binding Partner can be encoded to be expressed in fusion to a protein or peptide that is not a desired protein of interest, such as an affinity tag.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows SDS PAGE analysis comparing of cell lysates of N-Intein Ligand produced by conventional single-product overexpression in E. coli.

FIG. 2 shows SDS PAGE analysis comparing conventional single product overexpression to co-expression with a Cognate Binding Partner.

FIG. 3 shows SDS PAGE analysis demonstrating that the Cognate Binding Partner can be altered or expressed with various fusion partners.

FIGS. 4A-4C show a comparison of Ligand solubility for conventional single-product overexpression vs. CBP co-expression batches. Each batch was expressed and processed in parallel under identical conditions. FIG. 4A shows SDS Page comparison. FIG. 4B shows retention volume in conventional vs. Ligand and CBP processing. FIG. 4C shows elution peaks for normalized yield.

FIG. 5 shows SDS PAGE analysis showing end-use purification and cleaving kinetics assay. Resin used in lower panel was generated using methods disclosed herein.

FIGS. 6A-6C show a generalized modular structures of principle components comprising the disclosed invention. (FIG. 6A) Modular Structures of an N-Intein Ligand comprising a split intein segment and operably linked fusion partners. The ligand is comprised of an N-terminal intein segment (INT_{N}) at minimum, but may also be comprised of additional protein/peptide domains/motifs/moieties expressed as fusion partners with the INT_{N} segment. These fusion partners may include a Sensitivity Enhancing Motif (SEM), and various “Immobilization” Moieties (I), “Linker” Moieties (L), and/or “Tag” Moieties (T). (FIG. 6B) A Cognate Binding Partner (CBP), which minimally is defined as a Peptide/protein capable of binding INT_{N} counterpart to induce folded, stabilized state. The CBP may or not include optional tag and linker moieties expressed in fusion with either terminus. INTc segments and peptides derived from INTc species constitute a specific subset of CBP that may be used to induce INT_{N} stabilization. The term ‘Cognate Binding Partner’ is used because the intein complex resulting from association between an INT_{N} segment and CBP may not necessarily be capable of exhibiting cleaving or splicing activity; a subtle but important distinction from the more specific INT_{C} subset. (FIG. 6C) Generalized example of INT_{N} stabilization induced by a binding event between an INT_{N} segment and Cognate Binding Partner.

FIG. 7 shows a generalized process illustrating various standard heterologous expression techniques that could be used to produce an N-Intein Ligand that has been stabilized by a Cognate Binding Partner, for the purpose of manufacturing an intein-mediated capture medium.

FIGS. 8A-8B show a generalized manufacturing process comparing (FIG. 8A) ‘Conventional’ bioprocessing steps to (FIG. 8B) the manufacturing process claimed herein. Both processes produce an affinity capture medium comprising an immobilized N-Intein Ligand of identical sequence composition. Shown in the dotted box of each panel is ‘Active’ affinity capture media just before end-use as shown in the final “intein-mediated affinity capture” step. This illustrates and contrasts the critical differences in the manufacturing process necessitated by the introduction of the Cognate Binding Partner. Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIGS. 9A-9D illustrate a standard calculation basis for compression factor, peak asymmetry, and reduced plate height column efficiency metrics. (FIG. 9A) Illustration of measurement of bed compression factor during column packing procedures. (FIG. 9B) A generalized example of a tracer pulse injection test chromatogram. Tracer concentration (monitored by A₂₈₀) in the column effluent is plotted as a function of retention volume. Annotations have been added to illustrate and define parameters used to evaluate column efficiency. (FIG. 9C) List of relevant parameters and associated notation defined for terms used in evaluation of column packing and calculation of column efficiency metrics. (FIG. 9D) Definitions and expressions used to calculate column efficiency metrics.

FIGS. 10A-10B show column efficiency data from tracer pulse injection tests performed on two resin batches, packed with and without the aid of a Cognate Binding Partner (+CBP and -CBP, respectively), as described in Example 5. (FIG. 10A) Chromatograms overlaid from each batch, where UV absorbance in the column effluent (A₂₈₀) is plotted vs. retention time. (FIG. 10B) Bar graphs comparing column efficiency metrics for each batch, as calculated from the chromatogram data shown in FIG. 10A. To illustrate the effect that the Cognate Binding Partner has on column packing, FIG. 10B summarizes the critical column efficiency metrics - Cf, As, and h - which are reported for each batch. Also illustrated in FIG. 10B are the ideal and acceptable values/ranges for each metric (denoted by dotted lines and green shaded regions, respectively), which are provided for comparison to the values calculated from the experimental results for each batch.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

The term “contacting” as used herein refers to bringing two biological entities together in such a manner that the compound can affect the activity of the target, either directly; i.e., by interacting with the target itself, or indirectly; i.e., by interacting with another molecule, co-factor, factor, or protein on which the activity of the target is dependent. “Contacting” can also mean facilitating the interaction of two biological entities, such as peptides, to bond covalently or otherwise.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, troubleshooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “target protein”, “protein of interest” and “therapeutic agent” include any synthetic or naturally occurring protein or peptide. In the context of this invention, a “protein of interest” is a protein that is to be purified using split intein purification technology by an end user in a laboratory or manufacturing setting, as opposed to any context related to the manufacture of the purification medium itself. This definition would apply to any protein or peptide requiring purification for study or other research applications. The term additionally encompasses those compounds traditionally regarded as drugs, vaccines, and biopharmaceuticals including molecules such as proteins, peptides, and the like. Examples of therapeutic agents are described in well-known literature references such as the Merck Index (14th edition), the Physicians’ Desk Reference (64th edition), and The Pharmacological Basis of Therapeutics (1st edition), and they include, without limitation, medicaments; substances used for the treatment, prevention, diagnosis, cure or mitigation of a disease or illness; substances that affect the structure or function of the body, or pro-drugs, which become biologically active or more active after they have been placed in a physiological environment.

As used herein, “variant” refers to a molecule that retains a functional activity that is the same or substantially similar to that of the original sequence. The variant may be from the same or different species or be a synthetic sequence based on a natural or prior molecule. Moreover, as used herein, “variant” refers to a molecule having a structure attained from the structure of a parent molecule (e.g., a protein or peptide disclosed herein) and whose structure or sequence is sufficiently similar to those disclosed herein that based upon that similarity, would be expected by one skilled in the art to exhibit the same or similar activities and utilities compared to the parent molecule. For example, substituting specific amino acids in a given peptide can yield a variant peptide with similar activity to the parent.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, linkage of distinct domains or motifs, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins-Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, “isolated peptide” or “purified peptide” is meant to mean a peptide (or a fragment thereof) that is substantially free from the materials with which the peptide is normally associated in nature, or from the materials with which the peptide is associated in an artificial expression or production system, including but not limited to an expression host cell lysate, growth medium components, buffer components, cell culture supernatant, or components of a synthetic in vitro translation system. The peptides disclosed herein, or fragments thereof, can be obtained, for example, by extraction from a natural source (for example, a mammalian cell), by expression of a recombinant nucleic acid encoding the peptide (for example, in a cell or in a cell-free translation system), or by chemically synthesizing the peptide. In addition, peptide fragments may be obtained by any of these methods, or by cleaving full length proteins and/or peptides.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “isolated nucleic acid” or “purified nucleic acid” is meant to mean DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequences. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or peptide molecules.

“Intein” refers to an in-frame intervening sequence in a protein as described by Perler (Perler, Davis et al. 1994). An intein can catalyze its own excision from the protein through a post-translational protein splicing process to yield the free intein and a mature protein. An intein can also catalyze the cleavage of the intein-extein bond at either the intein N-terminus, or the intein C-terminus, or both of the intein-extein termini. As used herein, “intein” encompasses mini-inteins, modified or mutated inteins, and split inteins.

The term “Split Intein” refers to a pair of two distinct and separately translated protein segments, comprising an “N-Terminal Intein Segment” (INT_{N}) and a counterpart “C-Terminal Intein Segment” (INT_{C}) binding partner, which are characterized by at least one of the following properties:
(1) INT_{N} and INT_{C} segments exhibit an innate affinity for their respective counterpart protein, which drive the pair to spontaneously associate, fold, and non-covalently “bind” together, forming an “Intein Complex”.
(2) Upon association, an Intein Complex may become “Splicing Active” or “Cleaving Active”, wherein the complex catalyzes cleaving or splicing events between the complex and its extein fusion partners. This activity is generally considered to be contingent upon formation of the Intein Complex, which is to say that neither INT_{N} nor INT_{C} posses said activity autonomously in the absence of their binding partner.
(3) INT_{N} and INT_{C} segments containing peptides, protein domains, or amino acid sequences that are identical, similar to, or derived from naturally occurring or artificially split inteins, such as those cataloged in the so-called “InBase, The Intein Database” established by Perler (Perler 1999, Perler 2002). Examples of intein species are also listed in Table 2.
(4) It should be noted though that the formation of complexes exhibiting cleaving and/or splicing activity is not strictly required to satisfy the definition of “Split Intein” and/or INT_{N} and/or INT_{C} segments. In other words, for example, if a “Split Intein” has been modified so that it no longer possesses the characteristic of exhibiting splicing and/or cleaving activity, it is still encompassed by this invention.

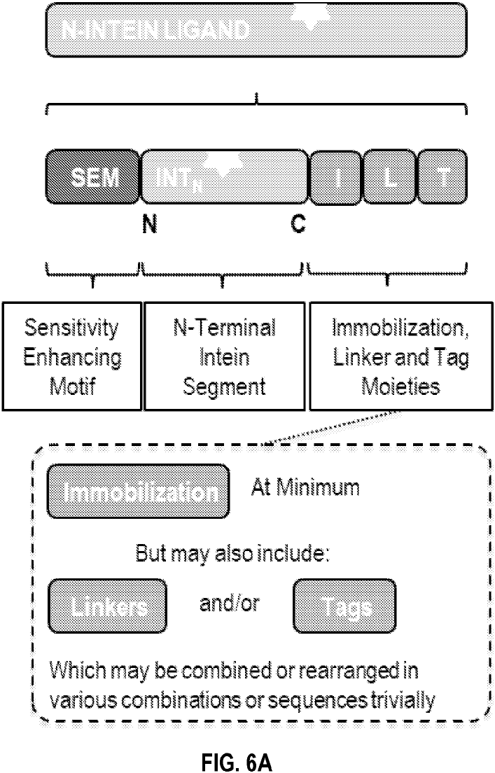

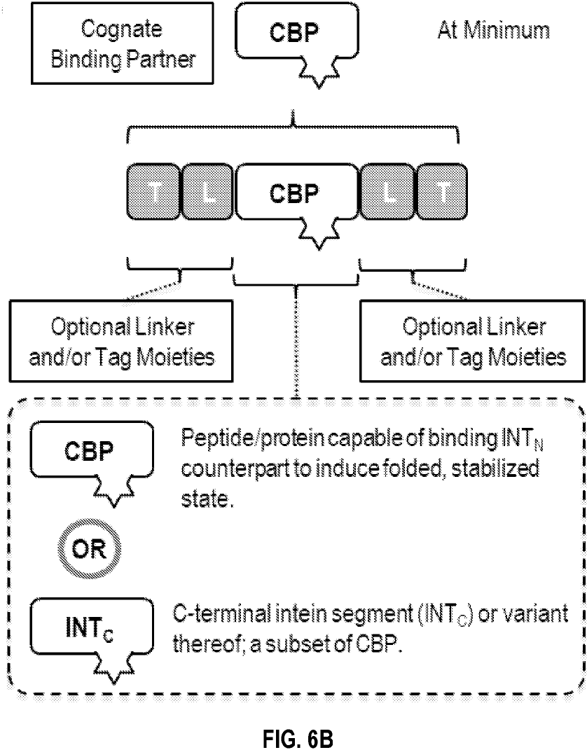

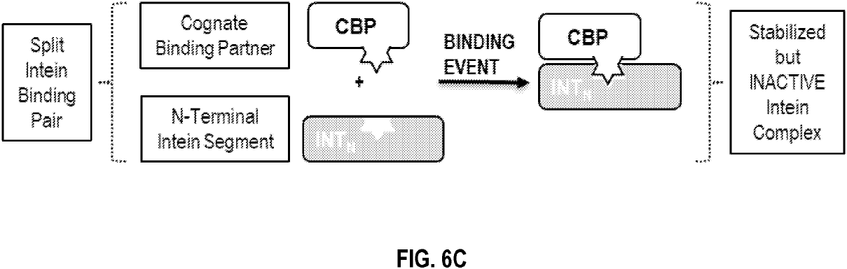

The term “Cognate Binding Partner” or “Cognate” refers to any peptide or protein segment capable of spontaneous, non-covalent association with any “Binding Active” INT_{N} counterpart it contacts. Cognate Binding Partners include, but are not limited to, the subset of peptides and protein segments that comprise species defined as INT_{C} peptides, including INT_{C} peptides that have been operably linked to additional linker and tag moieties as shown in FIG. 6(b) and described below. For example, an INT_{C} segment may be an example of a Cognate Binding Partner, but a Cognate Binding Partner is not by definition strictly required to be a species of INT_{C}.

INT_{C} are also herein further differentiated from the Cognate superfamily in that INT_{C} are specifically those Binding Partners that associate with INT_{N} to form an ACTIVE Intein Complex.

INT_{C} should be considered a Cognate if it associates with INT_{N} and folds into an Intein Complex, but the resulting complex is an INACTIVE Intein Complex (exhibits no splicing or cleaving activity).

As used herein, the term “Extein” refers to any peptide, protein, domain, or amino acid that is expressed covalently in fusion to either the N-terminus of an INT_{N} segment, the C-terminus of an INT_{C} segment. Exteins are further characterized as the portion of said intein-fused polypeptide which may be cleaved or spliced upon excision of the intein or intein complex.

The N-terminal Extein (N-EXT) is specifically the Extein expressed in fusion with the N-terminus of the INT_{N} segment. An N-EXT is only classified as such if expressed in fusion with an INT_{N} segment, however, an INT_{N} segment does not strictly require the presence of an N-EXT to satisfy the definition of INT_{N} segment.

The C-terminal Extein (C-EXT) is specifically the Extein expressed in fusion with the C-terminus of an INT_{C} segment or cognate binding partner. A C-EXT is only classified as such if expressed in fusion with an INT_{C} segment or cognate binding partner, however, INT_{C} segments and cognate binding partners do not strictly require the presence of a C-EXT to satisfy their respective definitions.

Furthermore, N-EXT and C-EXT domains may continue to be identified as such after cleaving or splicing events occur, despite being excised from their respective INT_{N} and INTc fusion partners.

The term “N-Intein Ligand” refers to a protein that has been (or will be) immobilized onto a solid surface, substrate or chromatographic medium to function as an affinity ligand. As defined herein, the N-Intein Ligand is comprised of an INT_{N} segment at minimum, but may also be comprised of additional operably linked proteins, peptides, functional domains, amino acid motifs and or chemical moieties, which are expressed as fusion partners with the INT_{N} segment (FIG. 6). Fusion partners that comprise the N-Intein Ligand may include (but are not limited to) a Sensitivity Enhancing Motif (SEM), as well as various “Immobilization Moieties”, “Linker Moieties”, and/or “Tag Moieties”, which collectively are referred to as “ILT Moieties”.

The term “Sensitivity Enhancing Motif” (SEM) refers to an amino acid sequence of three or more residues expressed in fusion with the N-terminus of an INT_{N} segment, which renders the splicing or cleaving activity of an intein complex highly sensitive to extrinsic conditions as described previously in U.S. Pat. 10,066,027. The SEM is a constitutive element of an N-Intein Ligand, but is distinct from the INT_{N} segment and other fusion partners that may comprise said N-Intein Ligand.

“ILT Moieties” is a collective term for one or more amino acids expressed as fusion partners with an INT_{N} to comprise an N-Intein Ligand. ILT moieties can be further subdivided into constituent groups that include at least one of the “immobilization” (I), “linker” (L), and/or “tag” (T) moiety classifications that are defined further below. individual moieties are operably linked, and may be trivially repeated, combined or rearranged in relation to each other, and in relation to the INT_{N} (for examples see FIG. 6).

The term “immobilization moiety” refers to one or more amino acid residues (e.g. Cys), expressed in fusion with the INT_{N}, which allows for covalent immobilization of the N-Intein Ligand (and its fusion partners by extension).

The classification “linker moiety” or “linker” refers to one or more amino acid residues expressed in fusion with the INT_{N} that confers structure, spacing, or flexibility between the INT_{N}, the immobilization moiety, and/or other fusion partners. Common examples of linker moieties include, but are not limited to: Glycine-Serine repeat ((Glyₙ₁Serₙ₂)ₙ₃), Polyproline dyad ((XaaPro)ₙ), and α-helical (A(EAAAK)ₙA) linker motifs.

The classification “tag moiety” or “tag” refers to a peptide, domain, or a specific amino acid motif that is expressed in fusion with a protein, and aids in purification, detection, and/or enhances soluble expression of its fusion partners. Examples of common “tag” moieties include but are not limited to: purification tags (e.g. poly-His, poly-Arg, GST, CBD, MBP, CBP, Strep-Tag, FLAG-tag, etc.), detection tags (e.g. GFP, luciferase, epitope tags (i.e. FLAG, HA, c-myc), HRP, etc.), and expression/solubility enhancing tags (e.g. T7-tag, NusA, TrxA, DsbA, DsbC, GST, MBP, etc.).

An INT_{N}, INT_{C} or Cognate Binding Partner domain is considered “Binding Active” if the segment exhibits affinity for its counterpart binding partner and can participate in a Binding Event that forms a new Intein Complex. The terms “Binding Active” and “Binding Inactive” are used to distinguish functional, singular INT_{N}, INT_{C} and/or Cognate segments from otherwise compositionally identical segments, which have (a) already bound a partner to form an an Intein Complex, or (b) misfolded in such a way as to suppress the segment’s affinity for its potential binding partners. Importantly, when comprising an Intein Complex, constituent INT_{N}, INT_{C} and/or Cognate segments can bind each other such that they cannot further associate with additional otherwise compatible binding partners that they might encounter while the Intein Complex exists. For example, a given INT_{N} and INT_{C} may associate and bind each to form an Intein Complex, but upon formation of said complex, the INT_{N} and INT_{C} can become functionally “Binding Inactive” - neither segment can participate in any further binding events while comprising the Intein Complex. However, if the Intein Complex is dissolved, and the INT_{N} and INT_{C} are dissociated and subsequently refolded such that their affinity is restored, the individual segments may again become “Binding Active”.

An Intein Complex can be further functionally classified as either “INACTIVE” or “ACTIVE” with respect to intein splicing and/or cleaving activity. An INACTIVE Intein Complex is one where the Intein Complex exhibits less than 10% cleaving or splicing behavior with its Extein fusion partners. Conversely, An ACTIVE Intein Complex is one where the catalyze a cleaving or splicing event that alters the peptide bonds of at least one of its Extein fusion partners.

An ACTIVE Intein Complex may be further categorized by the specific type of canonical intein event that it catalyzes: C-Terminal Cleaving, N-Terminal Cleaving, Dual Cleaving, or Splicing.

Once an “Active Intein Complex” catalyzes a cleaving or splicing event, the resulting Intein Complex may have no further effect on the peptide bonds of its fusion partners (splicing and cleaving reactions are irreversible), and thus the resulting Intein Complex can generally be considered an “INACTIVE Intein Complex” after catalyzing any cleaving or splicing event. By “no further effect” is meant less than a 10% effect.

As used herein, the term “splice” or “splices” means to excise a central portion of a polypeptide to form two or more smaller polypeptide molecules. In some cases, splicing also includes the step of fusing together two or more of the smaller polypeptides to form a new polypeptide. Splicing can also refer to the joining of two polypeptides encoded on two separate gene products through the action of a split intein.

As used herein, the terms “cleave”, “cleaves”, “cleavage” and “a cleaving event” refer to a chemical reaction in which a peptide bond within a polypeptide is broken, thereby dividing a single polypeptide to form two or more smaller polypeptide molecules. In some cases, cleavage is mediated by the addition of an extrinsic endopeptidase, which is often referred to as “proteolytic cleavage”. In other cases, cleaving can be mediated by the intrinsic activity of one or both of the cleaved peptide sequences, which is often referred to as “self-cleavage”. Cleavage can be controlled by extrinsic conditions (such as buffer pH), as in the action of the split intein system described herein.

By the term “fused” or “in fusion with” is meant covalently bonded to. For example, a first peptide is fused to a second peptide when the two peptides are covalently bonded to each other (e.g., via a peptide bond). Peptides and/or protein domains conjoined by peptide bonds may also be referred to as “fusion partners”.

As used herein an “isolated” or “substantially pure” substance is one that has been separated from components which naturally accompany it. Typically, a polypeptide is substantially pure when it is at least 50% (e.g., 60%, 70%, 80%, 90%, 95%, and 99%) by weight free from the other proteins and naturally-occurring organic molecules with which it is naturally associated.

Herein, “bind”, “binds”, “binding” or “binding event” means that one molecule recognizes and adheres to another molecule in a sample, but does not substantially recognize or adhere to other molecules in the sample. The terms “bind”, “binds”, “binding” and “binding event” also imply the interaction between two molecules is non-covalent and reversible. One molecule “specifically binds” another molecule if it has a binding affinity greater than about 10⁵ to 10⁶ liters/mole for the other molecule. These terms are used interchangeably with “associate with,” “associates with,” or “associating with.”

Nucleic acids, nucleotide sequences, proteins or amino acid sequences referred to herein can be isolated, purified, synthesized chemically, or produced through recombinant DNA technology. All of these methods are well known in the art.

As used herein, the terms “modified” or “mutated,” as in “modified intein” or “mutated intein,” refer to one or more modifications in either the nucleic acid or amino acid sequence being referred to, such as an intein, when compared to the native, or naturally occurring structure. Such modification can be a substitution, addition, or deletion. The modification can occur in one or more amino acid residues or one or more nucleotides of the structure being referred to, such as an intein.

As used herein, “operably linked” refers to the association of two or more biomolecules in a configuration relative to one another such that the normal function of the biomolecules can be performed. In relation to nucleotide sequences, “operably linked” refers to the association of two or more nucleic acid sequences, by means of enzymatic ligation or otherwise, in a configuration relative to one another such that the normal function of the sequences can be performed. For example, the nucleotide sequence encoding a pre-sequence or secretory leader is operably linked to a nucleotide sequence for a polypeptide if it is expressed as a pre-protein that participates in the secretion of the polypeptide; a promoter or enhancer is operably linked to a coding sequence if it affects the transcription of the coding sequence; and a ribosome binding site is operably linked to a coding sequence if it is positioned so as to facilitate translation of the sequence.

“Sequence homology” can refer to the situation where nucleic acid or protein sequences are similar because they have a common evolutionary origin. “Sequence homology” can indicate that sequences are very similar. Sequence similarity is observable; homology can be based on the observation. “Very similar” can mean at least 70% identity, homology or similarity; at least 75% identity, homology or similarity; at least 80% identity, homology or similarity; at least 85% identity, homology or similarity; at least 90% identity, homology or similarity; such as at least 93% or at least 95% or even at least 97% identity, homology or similarity. The nucleotide sequence similarity or homology or identity can be determined using the “Align” program of Myers et al. (1988) CABIOS 4:11-17 and available at NCBI. Additionally or alternatively, amino acid sequence similarity or identity or homology can be determined using the BlastP program (Altschul et al. Nucl. Acids Res. 25:3389-3402), and available at NCBI. Alternatively or additionally, the terms “similarity” or “identity” or “homology,” for instance, with respect to a nucleotide sequence, are intended to indicate a quantitative measure of homology between two sequences.

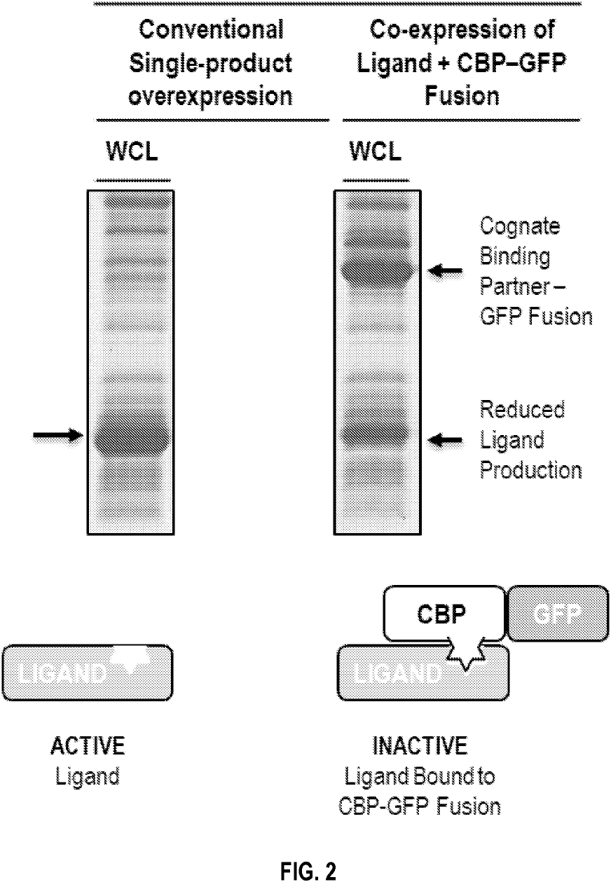

Alternatively or additionally, “similarity” with respect to sequences refers to the number of positions with identical nucleotides divided by the number of nucleotides in the shorter of the two sequences wherein alignment of the two sequences can be determined in accordance with the Wilbur and Lipman algorithm. (1983) Proc. Natl. Acad. Sci. USA 80:726. For example, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity with DNA sequences, thymidine (T) in the DNA sequence is considered equal to uracil (U) in the RNA sequence. The following references also provide algorithms for comparing the relative identity or homology or similarity of amino acid residues of two proteins, and additionally or alternatively with respect to the foregoing, the references can be used for determining percent homology or identity or similarity. Needleman et al. (1970) J. Mol. Biol. 48:444-453; Smith et al. (1983) Advances App. Math. 2:482-489; Smith et al. (1981) Nuc. Acids Res. 11:2205-2220; Feng et al. (1987) J. Molec. Evol. 25:351-360; Higgins et al. (1989) CABIOS 5:151-153; Thompson et al. (1994) Nuc. Acids Res. 22:4673-480; and Devereux et al. (1984) 12:387-395. “Stringent hybridization conditions” is a term which is well known in the art; see, for example, Sambrook, “Molecular Cloning, A Laboratory Manual” second ed., CSH Press, Cold Spring Harbor, 1989; “Nucleic Acid Hybridization, A Practical Approach”, Hames and Higgins eds., IRL Press, Oxford, 1985; see also FIG. 2 and description thereof herein wherein there is a sequence comparison.

The terms “plasmid” and “vector” and “cassette” refer to an extrachromosomal element often carrying genes which are not part of the central metabolism of the cell and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Typically, a “vector” is a modified plasmid that contains additional multiple insertion sites for cloning and an “expression cassette” that contains a DNA sequence for a selected gene product (i.e., a transgene) for expression in the host cell. This “expression cassette” typically includes a 5′ promoter region, the transgene ORF, and a 3′ terminator region, with all necessary regulatory sequences required for transcription and translation of the ORF. Thus, integration of the expression cassette into the host permits expression of the transgene ORF in the cassette.

The term “buffer” or “buffered solution” refers to solutions which resist changes in pH by the action of its conjugate acid-base range.

The term “loading buffer” or “binding buffer” refers to the buffer containing the salt or salts which is mixed with the protein preparation for loading the protein preparation onto a column. This buffer is also used to equilibrate the column before loading, and to wash to column after loading the protein.

The term “wash buffer” is used herein to refer to the buffer that is passed over a column (for example) following loading of a protein of interest (such as one coupled to a C-terminal intein fragment, for example) and prior to elution of the protein of interest. The wash buffer may serve to remove one or more contaminants without substantial elution of the desired protein.

The term “elution buffer” refers to the buffer used to elute the desired protein from the column. As used herein, the term “solution” refers to either a buffered or a non-buffered solution, including water.

The term “washing” means passing an appropriate buffer through or over a solid support, such as a chromatographic resin.

The term “eluting” a molecule (e.g. a desired protein or contaminant) from a solid support means removing the molecule from such material.

The term “contaminant” or “impurity” refers to any foreign or objectionable molecule, particularly a biological macromolecule such as a DNA, an RNA, or a protein, other than the protein being purified, that is present in a sample of a protein being purified. Contaminants include, for example, other proteins from cells that express and/or secrete the protein being purified.

The term “separate” or “isolate” as used in connection with protein purification refers to the separation of a desired protein from a second protein or other contaminant or mixture of impurities in a mixture comprising both the desired protein and a second protein or other contaminant or impurity mixture, such that at least the majority of the molecules of the desired protein are removed from that portion of the mixture that comprises at least the majority of the molecules of the second protein or other contaminant or mixture of impurities.

The term “purify” or “purifying” a desired protein from a composition or solution comprising the desired protein and one or more contaminants means increasing the degree of purity of the desired protein in the composition or solution by removing (completely or partially) at least one contaminant from the composition or solution.

The terms “chromatography media” or “chromatographic medium” refer to any type of stationary phase substrate (solid support), scaffold, or matrix used for chromatography or purification, in which a N-Intein Ligand is affixed, immobilized, bonded, or grafted (covalently or otherwise), for the purpose of separating, enriching, or purifying a secondary molecule of interest. Common examples of chromatography media include but are not limited to: chromatography resins (e.g. crosslinked agarose, polymer, or silica-based particles/porous beads); functionalized membranes; micro- and nano-scale magnetic particles; and structured pore/structured channel media (e.g. monoliths and monolithis columns).

Disclosures herein relating to immobilization of a N-Intein Ligand upon a “chromatographic medium” are presumed to apply generally to any type of “chromatography media”. The fundamental functional requirement of the “chromatographic medium” is to provide a solid support surface to retain a N-Intein Ligand. As such, it is understood that various chromatographic media may be freely and independently substituted for one another with little or no consequence upon the function of the immobilized N-Intein Ligand.

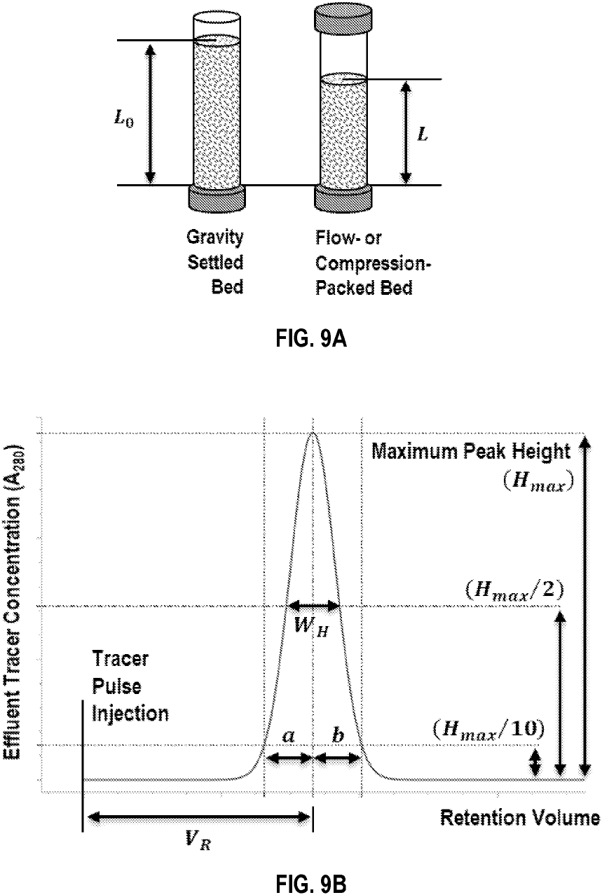

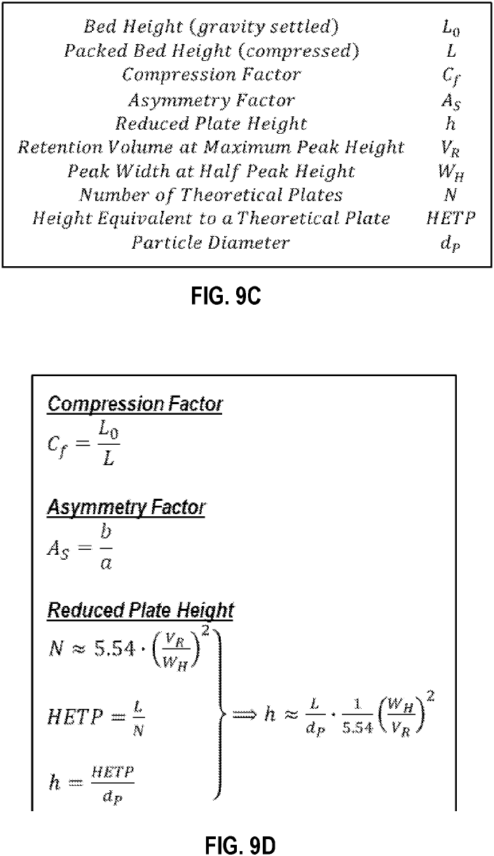

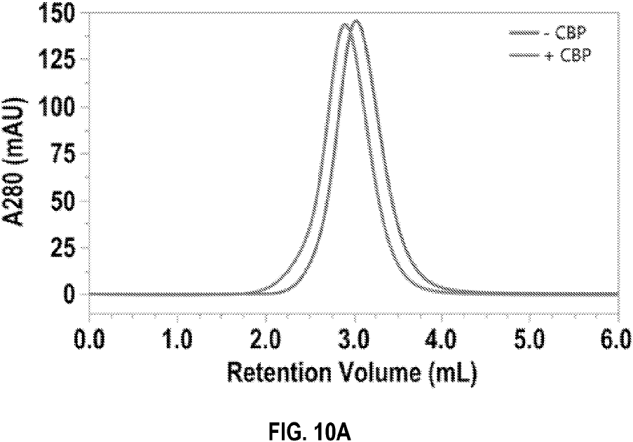

The term “asymmetry factor” denoted by the symbol “As”, refers to a column efficiency metric used to assess uniformity of flow through a packed-bed chromatography column. The asymmetry factor is determined with data collected by a standard column efficiency test conducted with a tracer pulse injection, then calculated using the expressions and definitions illustrated in FIG. 9.

The term “reduced plate height” denoted by the symbol “h”, refers to a column efficiency metric based on theoretical plate height, normalized to particle size within a packed-bed chromatography column. The reduced plate height is determined with data collected by a standard column efficiency test conducted with a tracer pulse injection, then calculated using the expressions and definitions illustrated in FIG. 9.

The term “column efficiency metrics” refer collectively to the asymmetry factor (As) and reduced plate height (h) which are standard metrics commonly cited to judge the quality of packing and uniformity of flow through a packed-bed chromatography column.

The term “compression factor” denoted by the symbol “C_{f}”, refers to the relative change in volume that a compressible chromatography resin will experience when being packed into a chromatography column. A common definition used in industry and those skilled in the art, compression factor is typically calculated by the expression (C_{f} = V_{expanded} / V_{compressed}); where V_{expanded} represents the volume of resin solids when fully expanded or “gravity settled”, and V_{compressed} represents the volume occupied by the same resin solids once they have been compressed in a packed resin bed. For columns with a constant cross-sectional area, this expression may be reduced to C_{f} = Lo / L, where L₀ is the height of a resin bed when fully expanded or “gravity settled”, and L is the height of the same resin bed when compressed, as illustrated in FIG. 9(a).

The term “sufficiently well packed” refers to a state of chromatography column packing in which the compression factor (C_{f)}, asymmetry factor (Aₛ), and reduced plate height (h) have ALL been measured to within their respective acceptable ranges.

The column efficiency metrics and definition of “sufficiently well packed” described above are universally recognized in the industry and are well established by those who are skilled in the art.

The term “intrinsic functional compressibility factor”, also abbreviated “IFCF”, refers to a property of a chromatography resin that indicates fractional volume change that a resin undergoes when packed to a chromatography column, relative to standardized packing conditions. IFCF is essentially a measurement of compression factor (C_{f}) that further stipulates a ‘standardized basis’ measurement method, which is necessary to ensure that the observed bed compression represents an exclusively intrinsic property of the resin. As defined herein, IFCF is the calculated compression factor (C_{f)} achieved when a resin is packed to a chromatography column in a manner that statisfies all the following ‘standardized basis’ conditions: (1) The resin must be suspended as a slurry and packed in phosphate buffered saline (PBS). (2) The packed resin bed generated during column packing must exhibit an asymmetry factor (As) between 0.8 and 1.4. (3) The packed resin bed generated during column packing must exhibit a reduced plate height (h) of less than 5.0 For example, if a resin was suspended as a slurry in PBS then allowed to gravity-settle in a chromatography column to a bed volume of X, and was then compressed to generate a packed resin bed volume of Y, then the packed resin bed is said to have a compression factor of C_{f} = X/Y. If subsequent column efficiency tests are then performed that verify the packed resin bed’s asymmetry factor and reduced plate height satisfy conditions (2) and (3) (e.g. an asymmetry factor of As = 1.0 and a reduced plate height h =3.0), then the resin’s intrinsic functional compressibility factor would be said to be IFCF = C_{f} = X/Y, as all ‘standard basis’ conditions were satisfied when the resin bed was packed.

In a second example, consider the same gravity-settled resin bed, which is instead packed with excessive compression, resulting in a smaller packed bed volume of Z as the resin’s porous, semi-elastic particle structure is crushed. This resin bed has a calculated compression factor of C_{f} = X/Z, despite being generated from the same resin as the previous example. Comparing these scenarios, it should be evident that compression factor (C_{f}) is specific to a given packed bed - the volumes Y and Z are partially determined by the intrinsic compressibility of the resin, but Y will differ from Z with variation in compressive packing force, which is both extrinsic and arbitrary. Therefore, a basis is specified to nomalize the compressive force applied during packing, so that any further deviations in compression are exclusively dependent on the resin’s intrinsic compressibility. Conditions (2) and (3) provide this standardized basis, since excessive (or insufficient) compression in the preparation of a packed bed will create irregular flow dynamics, which manifest as deviations in asymmetry factor (As) and/or reduced plate height (h). Indeed, asymmetry factor (As) and reduced plate height (h) will only satisfy conditions (2) and (3) when the degree of compression applied to the bed during packing is functionally appropriate for the mechanical structure of a given resin. In the second example, the resin bed was packed with an inappropriate amount of compression, and would therefore exhibit a poor asymmetry factor (As) and/or reduced plate height (h) (e.g. As = 0.6 or As = 1.8, and/or h = 6.5), thereby failing to satisfy the ‘standardized basis’ stipulations. Accordingly then, this packed resin bed’s measured compression factor of C_{f} = X/Z should not be considered a valid measure of the resin’s IFCF.

Likewise, resins are often slurried and packed in buffers of various compositions, but given that alternative buffer compositions are acknowledged to swell or shrink porous resins to various degrees, measuring resin compressibility from packed beds prepared with other buffers may lead to differing observations of compression factor (C_{f}). Therefore, it is necessary to specify the basis that measurements of IFCF be made in PBS buffer, which ensures that any deviations in measured compression are exclusively due to differences in resin composition that affect the resin’s intrinsic compressibility.

It should be understood that when the three ‘standard basis’ stipulations of the IFCF are met, the measured compression factor reflects an intrinsic property of the resin itself. Therefore, variations in IFCF may be used as an indirect method to detect changes in the resin’s composition.

The term “base resin” refers to the resin support substrate which has not had an N-Intein Ligand or any other ligand attached to it.

The term “compressibility differential” denoted by the symbol “ΔC” refers to the relative change in compressibility that a given resin may exhibit when a ligand is attached to a chromatography resin. Compressibility differential calculates the percentage difference between the intrinsic functional compressibility factor (IFCF) of a resin bearing an attached ligand, and that of its base resin substrate (IFCF_{BASE}). As defined herein, compressibility differential is calculated: ΔC = | (IFCF) - (IFCF_{BASE}) | / (IFCF_{BASE}) x 100%. For example, using the data presented in Example 5, the compressibility differential for the “-CBP″ resin batch would be calculated as ΔC = | (1.01) - ( 1.15) | / (1.15) x 100% = 12.2%, implying that the compressibility of the resin changed by more than 12% as a result of attaching N-Intein Ligand to the resin in the production of the “-CBP” batch. The resin’s compressibility differential (ΔC) can be less than about 1, 2, 3, 4, 5, 6, 7, 8, 9, 10, 11, 12, 13, 14, 15, 16, 17, 18, 19, or 20%, relative to its base resin substrate.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result. For example, compounds used to control pH in the examples shown can be substituted with other buffering compounds to control pH, since pH is the critical variable to be controlled and the specific buffering compounds can vary.

### B. Methods of Immobilizing N-Terminal Intein Segments

Intein-based methods of protein modification and ligation have been developed (U.S. Pat. 10,066,027 and U.S. Pat. 9,796,967, herein incorporated by reference in their entirety). An intein is an internal protein sequence capable of catalyzing a protein splicing reaction that excises the intein sequence from a precursor protein and joins the flanking sequences (N- and C-exteins) with a peptide bond (Perler et al. (1994)). Hundreds of intein and intein-like sequences have been found in a wide variety of organisms and proteins (Perler et al. (2002); Liu et al. (2003)), they are typically 350-550 amino acids in size and also contain a homing endonuclease domain, but natural and engineered mini-inteins having only the ~140-aa splicing domain are sufficient for protein splicing (Liu et al. (2003); Yang et al. (2004); Telenti et al. (1997); Wu et al. (1998); Derbyshire et al. (1997)).

Both contiguous and split inteins have been adapted for protein purification applications (U.S. Pat. 10,066,027 and U.S. Pat. 9,796,967), wherein modified inteins are used to mediate affinity capture of a secondary protein of interest. Split inteins in particular are useful for such applications due to their dimeric structure, binding-dependent cleaving activity, and strong natural affinity between counterpart segments. However, split inteins also commonly suffer from low yield or poor solubility when produced using ‘conventional’ bioprocessing techniques (Shah, Dann et al. 2012). Indeed, the protein yield attained via conventional processing is often so poor that scalable manufacturing of split intein-based chromatography media may be prohibitively expensive, and therefore not economically viable.

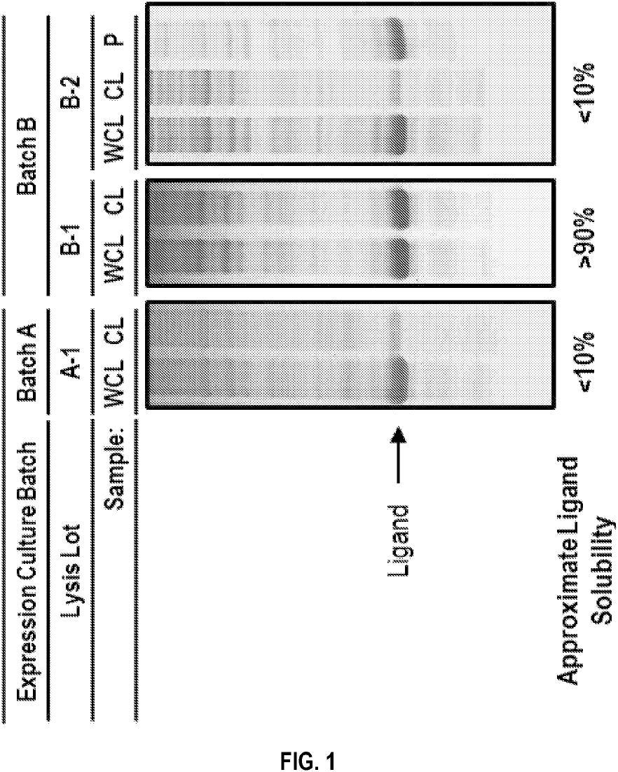

While production of any protein-based affinity ligand is certainly a complex multistep process involving many factors that influence overall yield, manufacturing bottlenecks are typically offset by upscaling the throughput-limiting unit operations. This approach appears to be particularly inefficient with split inteins, however, as solubility and aggregation are often the yield-limiting factors in the manufacturing process. Solubility in heterologous protein expression is typically regarded as a function of cell culture conditions and their impact on protein folding in vivo (e.g. proper formation of secondary and tertiary structures) (Rosano and Ceccarelli 2014) (Dyson and Wright 2005), split inteins however appear to be an exception to this view, as shown by the example in FIG. 1. Therefore, to improve manufacturing yields for split intein-based chromatography media, we have devised the novel processing techniques disclosed herein to mitigate stability issues specific to split inteins and their unique structure.

In the absence of their natural binding partners, INT_{N} and INT_{C} segments are primarily comprised of intrinsically disordered domains with little or no defined structural conformation (Zheng, Wu et al. 2012, Shah, Eryilmaz et al. 2013, Eryilmaz, Shah et al. 2014). This intrinsic disorder is putatively credited to explain the rapid, long-range, high-affinity binding exhibited between split intein segments (Pontius 1993, Shoemaker, Portman et al. 2000, Wright and Dyson 2009). While intrinsic disorder may confer the precise qualities that make split inteins amenable to affinity capture applications, it also implies that hydrophobic and charged residues within the disordered domain may be accessible or exposed, making split intein segments prone to aggregation and insolubility (Carrió and Villaverde 2002) (Saleh and Perler 2006) (Aranko, Wlodawer et al. 2014). Indeed, it was observed by Zheng et al. (2012), during fundamental studies on intein folding, that an INT_{N} segment from Synechocystis sp. PCC6803 was less soluble when expressed without its native INT_{C} counterpart, which the authors attribute to the ‘disordered’ structure of the isolated INT_{N} segment. The authors offer this observation in support of their hypothesis that inteins transition from disordered to folded states upon complex formation.

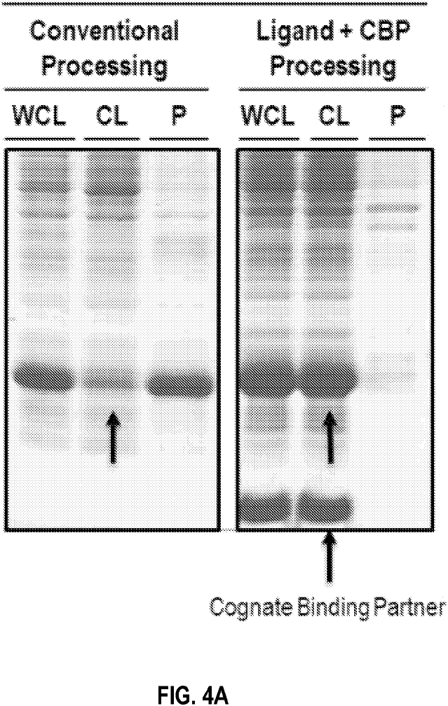

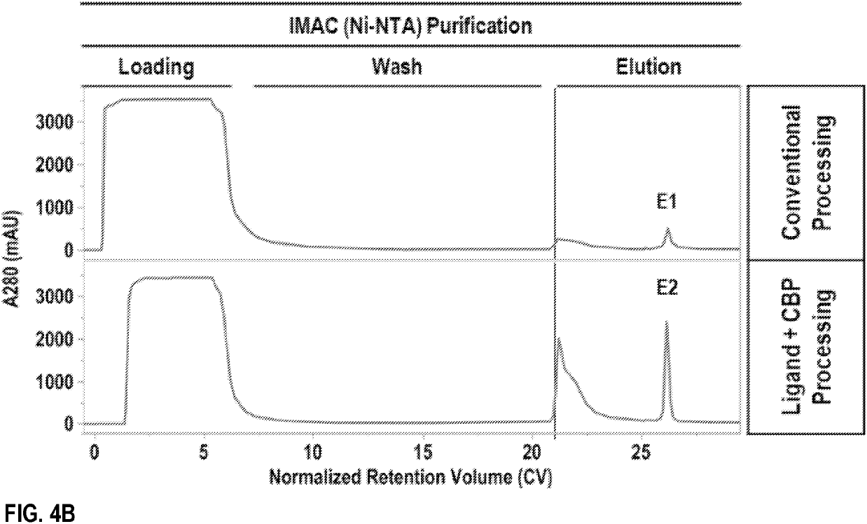

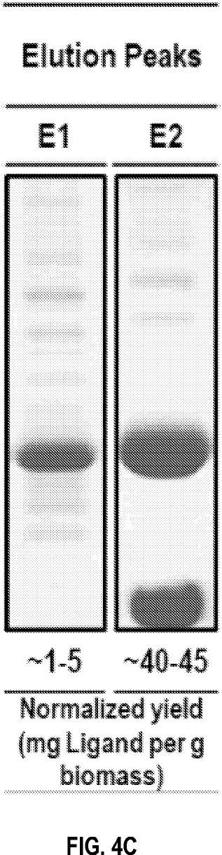

As claimed herein, an N-Intein Ligand may be stabilized during the manufacturing process by introducing a Cognate Binding Partner to induce a novel folded state that improves INT_{N} stability and solubility. This dramatically increases the overall manufacturing process yield, as demonstrated in the example shown in FIG. 4.

Importantly though, while the presence of the cognate binding partner improves process yield, it also functionally inactivates the INT_{N} segment, rendering the N-Intein Ligand incapable of binding or associating with any INT_{C}-fused proteins of interest that it might encounter. Given that the fundamental function of affinity capture media is predicated on its ability to bind a protein of interest, it is ostensibly counterintuitive to introduce excipient proteins that are known to deactivate the N-Intein Ligand during the manufacturing process.

Therefore, the feasibility of the disclosed manufacturing process is critically dependent on the ability to (1) dissociate the Cognate Binding Partner from the INT_{N} segment after covalent immobilization, and (2) revert the immobilized N-Intein Ligand to a binding-active folding state. Neither of these appear to have been previously demonstrated in the literature.

It is not clear that forced dissociation of split inteins is even possible without damaging their structure and/or activity in the process. The binding affinity between wild-type INT_{N} and INT_{C} segments have been measured in the low nanomolar range (Shi and Muir 2005) (Zettler, Schutz et al. 2009). This is likely an underestimate for split inteins that have been modified for affinity capture, as splicing exteins are unnecessary for this application and can therefore be eliminated to reduce steric binding inhibition. While it is understood that denaturants may be used to destabilize bound-protein complexes (O’Brien, Dima et al. 2007), stronger equilibrium binding affinities typically indicate significant energetic barriers to dissociation (Kastritis and Bonvin 2013). These barriers may be overcome using proportionally harsh denaturants, but this often cannot be achieved without incurring irreversible damage to the structure or activity of the protein components. Furthermore, several split inteins have been shown to resist even denaturing conditions, remaining complexed in the presence of denaturing chaotropes such as 6 M Urea (Southworth, Adam et al. 1998), as well as denaturing concentrations of detergents and reducing agents, such as 2% w/v SDS and 150 mM DTT (Nichols, Benner et al. 2003). Therefore, it may be logical to conclude that traditional approaches for stripping protein-based affinity ligands may fail to dissociate INT_{N} and INT_{C} segments. This might be overcome by treating an N-Intein Ligand with increasingly harsh denaturants, but risks damaging the intein structure and function irreversibly.

In addition to the binding reversibility concerns, it is non-trivial to design an immobilization reaction to selectively immobilize an N-Intein Ligand while it is complexed with a Cognate Binding Partner. The formation of the complex induces a restricted folding state in the N-Intein Ligand, which in turn may reduce accessibility to the reactive immobilization moiety within the ligand. Furthermore, the chemistries used to covalently immobilize proteins to a substrate may be reactive to both the N-Intein Ligand and the Cognate Binding Partner, resulting in the latter being grafted to the substrate.

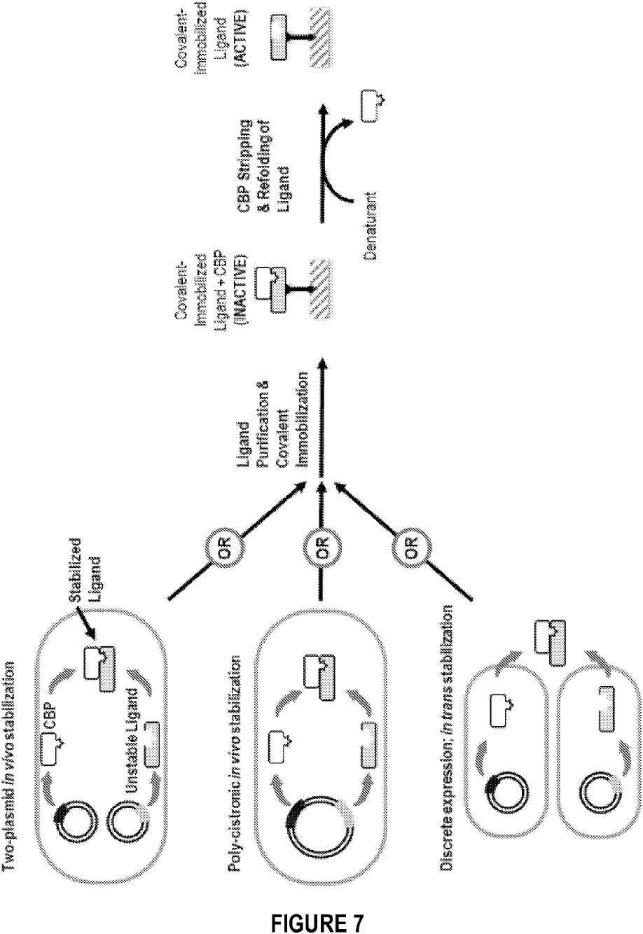

Even if a highly selective immobilization reaction can be designed, the Cognate Binding Partner is effectively consumed in the manufacturing process, and therefore incurs additional expense to produce. As shown in FIG. 7, a Cognate Binding Partner must either be expressed and purified separately and added to the N-Intein Ligand in trans, or co-expressed in cell culture with the N-Intein Ligand. The former requires a secondary production process for the Cognate Binding Partner - for which the added manufacturing expense should be obvious -while the latter option demonstrably reduces the expression titer of the N-Intein Ligand as shown by the example in FIG. 2.

It is worth noting though that solubility problems do not entirely preclude production of N-Intein Ligand using conventional manufacturing processes. Indeed, the compositions described in Millipore patent application WO 2016/073228 A1 and GE patent application US 2019/0263856 A1 imply that N-Intein Ligands can already be manufactured without the aid of a stabilizing Cognate Binding Partner. Clearly, an acceptable level of soluble product can be produced by conventional methods, which suggests that improving soluble yield should have only a modest impact on the overall productivity of the manufacturing process. For this reason, it was highly surprising to find that the Cognate Binding Partner enabled an order-of-magnitude improvement in yield, as shown in FIG. 4.

Considering the additional processing requirements that are created when stabilizing the N-Intein Ligand with a Cognate Binding Partner - (a) forcible dissociation of the intein complex without damage to the Ligand, (b) selective covalent immobilization of the Ligand in the presence of the Cognate, and (c) production of the Ligand at increased cost and/or reduced expression titer - it was unexpected to find that marginal increases in soluble yield could justifiably offset the barriers and expense incurred by introducing a Cognate Binding Partner during the manufacturing process.

In this method, expression of the N-Intein Ligand can take place in the presence of a Cognate Binding Partner, such as an INT_{C} segment. The Cognate Binding Partner and the N-Intein Ligand can be coexpressed in vivo, from a single or dual plasmid system, or the Cognate Binding Partner can be expressed in a separate cell and exposed to the N-Intein Ligand in trans, prior to downstream processing, as shown in FIG. 7. Due to the natural affinity between the N-Intein Ligand and the Cognate Binding Partner, the pair will spontaneously associate. This complex induces a ‘novel’ folding state that the N-Intein Ligand cannot adopt on its own, where the Cognate Binding Partner can shield specific hydrophobic and charged residues within the N-Intein Ligand that would otherwise drive nucleation events, aggregation, and insolubility. Via these steps, a functional intein capture medium is generated, which is capable of capturing a C-terminal intein tag for protein purification applications (e.g., as described in U.S. Pat. #10,066,027 B2).

The association of the intein complex (defined as the N-Intein Ligand associated with the Cognate Binding Partner) takes on a globular structure, which enhances protein stability by limiting the variety of conformations the N-Intein Ligand can adopt. This makes the N-Intein Ligand more resistant to degradation and/or aggregation during processing. For example, the intein complex can be 10, 20, 30, 40, 50, 60, 70, 80, or 90%, or one, two, three, four, or more orders of magnitude more soluble and/or resistant to degradation than an N-Intein Ligand not associated with a Cognate Binding Partner. Additionally, due to the increased structural and chemical stability of the N-Intein Ligand, the intein complex reduces the formation of product-related impurities associated with aggregation and degradation processes, and thereby confers greater physical and chemical homogeneity to the protein population than the N-terminal intein segment alone, which significantly simplifies downstream separation processes.

Furthermore, because the solubility of the folded intein complex is significantly greater than the N-Intein Ligand alone, it can be concentrated to significantly higher levels before and during the resin coupling reaction, which can improve N-Intein Ligand density during the immobilization process. For example, the intein complex can be 10, 20, 30, 40, 50, 60, 70, 80, or 90%, or one, two, three, four, or more orders of magnitude more soluble than the N-Intein Ligand alone, thus allowing N-Intein Ligand densities of greater than 10 mg ligand/mL resin bed volume. For example, the N-Intein Ligand density can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more mg ligand/mL resin bed volume.

Once the intein complex has been purified and concentrated, the N-terminal intein segment can be selectively covalently immobilized on a chromatographic media using standard bioconjugation techniques. This is discussed in more detail below. This selectivity is possible through several mutations engineered into the N-terminal intein segment (also discussed below). After immobilization, the N-terminal intein segment remains inactive for binding due to the induced folding state with the cognate folding partner. At this point, binding activity must be restored to the N-terminal intein segment for the resulting intein capture resin to become functional. This can be achieved by subjecting the immobilized intein complex to a strong chaotrope, strong acid, or strong base (e.g. 6 M guanidine hydrochloride, 150 mM phosphoric acid, or 0.5 M sodium hydroxide, respectively). It should be noted though that this can potentially be achieved using any other reagent or condition (e.g., heating) that can effectively denatures the N-Intein Ligand and/or disrupts association between the N-Intein Ligand and the Cognate Binding Partner, then be washed away or otherwise removed to leave behind immobilized N-Intein Ligand.

When referring to “washing away” the cognate folding partner with a chaotropic agent or acid, it is noted that, while the majority of cognate folding partners are removed using this method, it is possible that less than 1%, or 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, or 50% (or any amount less than or in-between these amounts) of Cognate Binding Partner may remain associated with the N-Intein Ligand. It is important to note that this Cognate Binding Partner is not expressed in fusion with a desired protein of interest, as discussed herein, but is instead a residual part of the manufacturing process.

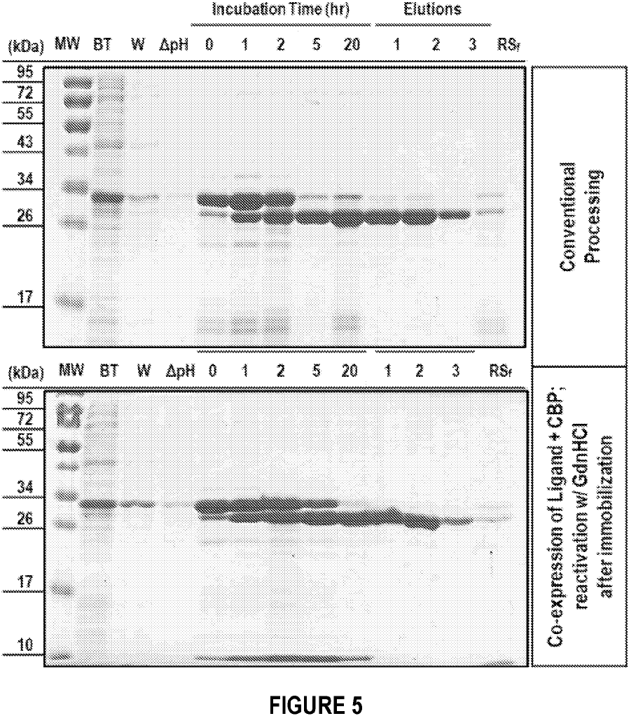

It is also noted that disrupting association between the N-Intein Ligand and the Cognate Binding Partner must be done in a way such that the N-Intein Ligand reverts to an active state, as opposed to being permanently inactivated by the denaturing condition. An example is shown in FIG. 5 (bottom panel), wherein the N-Intein Ligand accepts a new INT_{C} tagged protein of interest after disruption with Guanidine Hydrochloride. It is noted that “disrupting association between” means actively interrupting the association, or binding, of the N-Intein Ligand and the Cognate Binding Partner. This “stripping” or “disruption” of the cognate binding partner can be achieved by subjecting the immobilized intein complex to a chaotrope, strong acid, or strong base (e.g. guanidine hydrochloride, phosphoric acid, or sodium hydroxide, respectively), although this can potentially be achieved using any other reagent or condition (e.g., heating) that can effectively denature the N-Intein Ligand and/or disrupts association between the N-Intein Ligand and the Cognate Binding Partner.

While the primary motivation of the methods disclosed herein is to enhance solubility of the N-Intein Ligand, the stabilizing influence of the Cognate Binding Partner has been observed to have an unexpected and beneficial impact on packing the intein capture resin into a conventional chromatography column.

Column packing is an easily overlooked but nontrivial aspect of fixed bed liquid chromatography. Fixed bed packing quality can have a significant impact on separation efficiency and is crucial for consistent and reproducible performance. Uniform packing of the bed is vital for even distribution of fluid flow and consistent contact time throughout the column. Accordingly, improper packing can result in channeling, non-uniform mixing, irrregular contact time distribution, and/or underutilized fractions of the bed (Rathore, Kennedy et al. 2003). These issues effectively reduce separation efficiency and resolution, diminish product yield and purity, and may result in inconsistent performance and poor reproducibility. Unfortunately, when an N-Intein Ligand is conjugated to a particle-based chromatography substrate, the substrate’s bulk fluid behavior is altered in a way that makes intein capture resins exceptionally difficult to pack properly.

Particulate chromatography support substrates (i.e. resins made from cross-linked agarose, cellulose, dextran, polyacrylate, polystyrene, polyacrylamide, polymethacrylamide, or other polymers) are generally porous and compressible when subjected to moderate pressures, such as the differential pressure drop that develops across a chromatography column when operated. When packed with only gravity compression, a fixed bed comprised of these substrates will contract and expand as flow through the column is cycled on and off, respectively. Compression-relaxation cycles can damage the chromatography resins or reduce column performance by destabilizing the integrity of the packed bed, resulting in channeling, void formation, particle attrition, excessive backpressure, column dead-volume, non-uniform flow, and inconsistent residence time distributions . In order to avoid these issues, it is standard practice in the art to preemptively compress the chromatography media when it is packed into a column, then physically constrain the bed at a compressed volume to restrict potential reexpansion of the media. This is typically achieved either by flow-packing the resin as a slurry (i.e. pumping a slurry into a column at high flowrates to exceed the normal operating column pressure differential), and/or by applying mechanical compression directly to the resin bed axially. However, overcompression of a resin can also have damaging effects on column function, so different chromatography substrates are typically packed to a precisely defined compression range to ensure acceptable column performance.

The range of acceptable media compression is typically specified as a compression factor (C_{f}), expressed as a ratio of volumes: the volume of the fully-relaxed/expanded or “gravity settled” resin divided by the volume of the (compressed) resin bed within a packed column (C_{f} = V_{expanded} / V_{compressed}). The range of acceptable values for C_{f} may vary for different columns according to the matrix composition of the substrate and the diameter of the column being packed. Generally, substrate manufacturers specify an appropriate C_{f} based on empirical evaluation of the the base matrix and the pressures it is shown to tolerate. The majority of soft, porous matricies used in preparative bioprocessing require compression in the range of 1.10 < C_{f} < 1.15 for narrow-bore lab-scale columns, or 1.15 < C_{f} < 1.20 for large-diameter process-scale columns (Stickel and Fotopoulos 2001).

When a packed column is not sufficiently compressed to achieve a desired compression factor, it is trivial to apply additional mechanical or hydraulic pressure and further compress the bed to reach the specified C_{f} range. However, applying excessive force to the resin bed can crack, fracture, and/or crush the substrate particles. Evidence of overcompression or undercompression can often be detected by evaluating flow uniformity through a packed bed, so in addition to specifying a compression factor, it is common practice in the art to perform a standard column efficiency test to validate bed integrity after compressive packing is performed. Thus, a column is considered ‘sufficiently well packed’ only when BOTH the compression factor AND column efficiency metrics fall within specified ranges.

A common assay used to evaluate column efficiency is the tracer pulse injection test. Numerous variations of this methodology are described in the literature (Rathore, Kennedy et al. 2003, GE-Healthcare 2010, Andres, Broeckhoven et al. 2015), though all generally follow the consensus procedure performed by operating a column isocratically at constant flowrate, applying a pulse injection of an inert tracer, monitoring the column effluent as the tracer flows through the packed bed, then analyzing the tracer distribution to infer the quality and uniformity of column packing. The concentration of the tracer in the column effluent as a function of time is monitored continuously throughout the test and used to calculate standard column efficiency metrics - peak asymmetry factor (As) and reduced plate height (h) - using the relations and methodology illustrated in FIG. 9. Under ideal packing conditions, a column will have an asymmetry factor of As = 1.00 and a reduced plate height of h < 3. In practice, columns exhibiting an asymmetry factor in the range of 0.8 < As < 1.4 and a reduced plate height of h < 5 are generally regarded as satisfactory for column efficiency metrics. Column asymmetry factors of As < 0.8 are typically an indication of overpacking or excessive compression, while an asymmetry factor of As > 1.4 may indicate loose packing or bed instability.

For most porous particulate chromatography substrates, columns can be packed to the specified compression factor C_{f} while also satisfying the acceptable limits for column efficiency metrics As and h, regardless of the substrate particles’ functionalization or attached ligand composition. However, in an unexpected finding resulting from development of this work, particulate substrates were found to become far less compressible once an N-Intein Ligand had been conjugated to them. Given this phenomenon, it turns out to be exceedingly difficult - if not impossible - to achieve a sufficiently well packed resin bed when packing a column with an intein capture resin. Forturnately, the underlying mechanisms putatively responsible for reduced resin compressibility are similar to those believed to drive aggregation of the N-Intein Ligand, and can therefore similarly be mitigated by inclusion of a Cognate Binding Partner during the packing process, as shown in Example 5.

As previously noted, one of the defining characteristics of split inteins is the intrinsically disordered structure of the INT_{N} and INT_{C} domains when separated from their respective counterparts. In a disordered state, an intein’s hydrophobic and charged amino acid residues are exposed to the surrounding environment; intein association and binding is driven by these exposed residues, which attract and shield complementary residues in their counterpart domain, thereby folding together to form a more stable structured complex (Shah, Eryilmaz et al. 2013). While these exposed residues are essential to the functions that make split inteins useful for affinity capture, their inherent instability can also drive self-self interactions when concentrated, creating undesirable side effects. In addition to nucleating the INT_{N} domain aggregation responsible for the previously noted ligand solubility issues, it was found that this phenomenon also affects interactions between resin particles bearing surface-immobilized N-Intein Ligand. As shown in Example 5, the naturally compressible agarose base resin (C_{f}1.15) became incompressible (Ci=1.01) when conjugated with N-Intein Ligand. However, this effect was negated when the conjugated ligand was stabilized by the presence of a cognate binding partner, which restored the resin to its original pre-conjugation compressibility (C_{f}=1.15). The present invention therefore aids column packing, which is critical to the utility of the resin product.

INT_{C} segments expressed in fusion with a desired protein of interest are contemplated by this invention as part of a protein purification protocol, but it is noted that in this application they are not used until the N-Intein Ligand has already been covalently attached to a solid support and the Cognate Binding Partner has been removed. It is important to note that in this invention, similar INT_{C} segments are used both in the manufacturing and the intended end-use of the intein capture resin. The first time is as a cognate binding partner to protect the N-Intein Ligand and to promote its stability during the production of the intein capture resin and the packing of the intein capture resin into a conventional chromatogrtaphy column. This INT_{C} segment may have proteins or peptides associated with it, but it will not have a desired protein of interest (target protein, or protein that is desired as an end-product of this protein purification process). Once the N-Intein Ligand has been covalently conjugated to a solid support, the INT_{C} segment can be washed away by methods disclosed herein. After the N-Intein Ligand has been immobilized and reactivated by washing away the Cognate Binding Partner, the manufacturing process is essentially completed. At this point, during the intended end use of the resin, a second INT_{C} segment which comprises a desired protein of interest can be associated with the N-Intein Ligand during the purification of a desired protein of interest.

Both the INT_{N} and INT_{C} segments disclosed herein can be derived, for example, from an Npu DnaE intein.

The N-Intein Ligand, as defined herein can be derived from a native intein (such as Npu DnaE, for example; SEQ ID NO: 1), but can comprise additional modifications both within and outside of the canonically defined intein sequence. For example, the INT_{N} segment encoded by the Npu DnaE gene can be modified by conventional targeted mutagenesis so that it doesn’t comprise cysteine residues within the INT_{N} portion (SEQ ID NO: 2). It can also have additional amino acids appended to its N-terminus and/or C-terminus (defined as “within the N-terminal or C-terminal region) to improve cleaving performance and enable covalent immobilization onto a resin. This is described in detail above. A generalized structure of the N-Intein Ligand and its principle components are illustrated in FIG. 6(a).

In one example, the N-intein terminal segment can be modified so that at least one internal cysteine residue has been mutated to at least one serine residue, and a peptide sequence is appended to the C-terminus to enable simple purification and immobilization onto a resin, and a sensitivity enhancing peptide sequence is appended to the N-terminus to promote rapid and pH-sensitive cleaving (SEQ ID NO: 5 and see additional examples below). The fully modified sequence would be referred to as “the N-Intein Ligand” as described herein (SEQ ID NO: 5), and would comprise the Npu intein sequence and well as the described mutations and appended sequences.

The N-Intein Ligand can also comprise an immobilization moiety which allows for, or increases, covalent immobilization. For example, the one or more amino acids within the region of the C-terminus can be cysteine residues. This is desirous so as to eliminate side reactions associated with nonspecific immobilization of the N-Intein Ligand onto a solid support.

An example of an N-Intein Ligand in which the cysteine residues have been mutated can be found in SEQ ID NO: 2. It is noted that the first cysteine residue which is replaced (the first amino acid of the INT_{N} segment) can be replaced with either alanine or glycine so as to eliminate intein splicing in the assembled intein complex.

In the method disclosed herein, an intein complex stabilized by a Cognate Binding Partner can be immobilized onto a solid support substrate. A variety of supports can be used. For example, the solid support can be a polymer medium that allows for immobilization of the N-Intein Ligand, which can occur covalently or via an affinity tag with or without an appropriate linker. When a linker is used, the linker can be additional amino acid residues expressed in fusion with the N-Intein Ligand, or can be other known linkers for attachment of a peptide to a support.

The N-Intein Ligand disclosed herein can include an affinity tag as shown in FIG. 6(a). A linker sequence may also be utilized to create distance between the INT_{N} segment and affinity tag, while providing minimal steric interference to the intein cleaving active site. It is generally accepted that linkers involve a relatively unstructured amino acid sequence, and the design and use of linkers are common in the art of designing fusion peptides. There is a variety of protein linker databases which one of skill in the art will recognize. This includes those found in Argos et al. J Mol Biol 1990 Feb 20; 211(4) 943-58; Crasto et al. Protein Eng 2000 May; 13(5) 309-12; George et al. Protein Eng 2002 Nov; 15(11) 871-9; Arai et al. Protein Eng 2001 Aug; 14(8) 529-32; and Robinson et al. PNAS May 26, 1998 vol. 95 no. 11 5929-5934, hereby incorporated by reference in their entirety for their teaching of examples of linkers.

Table 1 shows exemplary sequences of the N-terminal intein segment and the C-terminal intein segment:

TABLE 1

| | | | |
|---|---|---|---|
| SEQ ID | Construct Name | Construct Name (Description) | Amino Acid Sequence |
| SEQ ID NO: 1 | Npu_{N}^{WT} | Wild-type Npu DNAE (INTrr segment) capable of splicing events | CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN |
| SEQ ID NO: 2 | Npu_{N}^{C1X} | Cleaving variant of SEQ ID NO: 1; cleaving phenotype resulting from a C1X mutation, where “X” = A or G | XLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN |
| SEQ ID NO: 3 | Npu_{N} ^{C1X,C-S} | Thiol-knockout variant of SEQ ID NO: 2, derived by mutating remaining Cysteine residues to Serine residues. | XLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPN |
| SEQ ID NO: 4 | S_{04b}- Npu_{N}^{C1X,C-S} | Variant of SEQ ID NO: 3 modified with Sensitivity-Enhancing Motif expressed as a fusion partner at the N-terminus | MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPN |
| SEQ ID NO: 5 | S_{04b}- Npu_{N}^{C1X,C-S}-G₄S-Hi_{S6}-Cys | Variant derived from SEQ ID NO: 4; constructed by adding linker, tag, and immobilization moiety fusion partners at the C-terminus of the N-Intein Ligand. | MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSHHHHHHC |
| SEQ ID NO: 6 | S_{04b}- Npu_{N}^{C1X,C-S}-G₄S-Cys-Hi_{S6} | Variant of SEQ ID NO: 5; derived from an alternate arrangement of the I-L-T fusion partners at the C-terminus. | MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCHHHHHH |
| SEQ ID NO: 7 | S_{04b}- Npu_{N}^{C1X,C-S}-G₄S-Cys- G₄S-Hi_{S6} | Variant of SEQ ID NO: 6 created by adding an additional linker between I-L-T moieties at the C-terminus. | MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCGGGGSHHHHHH |
| SEQ ID NO: 8 | S_{04b}- Npu_{N} ^{C1X,C-S}-(G₄S)₂- Cys-G₄S- Hi_{S6} | Variant of SEQ ID NO: 7 created by adding an additional linker between I-L-T moieties at the C-terminus. | MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSGGGGSCGGGGSHHHHHH |
| SEQ ID NO: 9 | S_{04b}- Npu_{N} ^{C1X,C-S}-(G₄S)₂- Cys-G₄S | Variant of SEQ ID NO: 8 created by removing the Hi_{S6} purification tag moiety from the C-terminus. | MGDGHGALSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSGGGGSCGGGGS |
| SEQ ID NO: 10 | Npu_{C}^{WT} | Wild-type Npu DNAE (INT_{C} segment) capable of splicing events | MIKIATRKYLGKQNVYDIGVERDHNFALKNGFIASN |
| SEQ ID NO: 11 | Npu_{C}^{D118G} | Cleaving variant of SEQ ID NO: 10; Accelerated cleaving phenotype resulting from D118G mutation | MIKIATRKYLGKQNVYGIGVERDHNFALKNGFIASN |
| SEQ ID NO: 12 | Npu_{C}^{DG,HN} | Variant derived from SEQ ID NO: 10 comprising D118G and S136H mutations, producing a cleaving phenotype with enhanced sensitivity to extrinsic conditions | MIKIATRKYLGKQNVYGIGVERDHNFALKNGFIAHN |
| SEQ ID NO: 13 | Npu_{C}^{DG,HN_} FFN-sfGFP-Hi_{S6} | Variant derived from SEQ ID NO: 12; A Cognate Binding Partner comprising a rapid-cleaving INT_{C} variant expressed with GFP and Hi_{S6} as fusion partner tag moieties | MIKIATRKYLGKQNVYGIGVERDHNFALKNGFIAHNFFNGTVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLEHHHHHH |
| SEQ ID NO: 14 | Npu_{C}^{DG,HA} | Variant of SEQ ID NO: 12, A Cognate Binding Partner modified with an N137A mutation that produces a binding-only (non-cleaving) phenotype | MIKIATRKYLGKQNVYGIGVERDHNFALKNGFIAHA |
| SEQ ID NO: 15 | Hi_{S6}-Npu_{C}^{DG,HA} | Variant of SEQ ID NO: 14, A non-cleaving Cognate Binding Partner expressed with a Hi_{S6} purification tag as an N-terminal fusion partner | MHHHHHHIKIATRKYLGKQNVYGIGVERDHNFALKNGFIAHA |
| SEQ ID NO: 16 | Npu_{C}^{DG,HA}- Hi_{S6} | Variant of SEQ ID NO: 14, A non-cleaving Cognate Binding Partner expressed with a Hi_{S6} purification tag as an C-terminal fusion partner | MIKIATRKYLGKQNVYGIGVERDHNFALKNGFIAHAHHHHHH |
| SEQ ID NO: 17 | Npu_{C}^{DG,HN_} MFN-sfGFP-Hi_{S6} | An INT_{C}-POI fusion construct for testing split intein-mediated affinity capture with a target protein of interest (sfGFP) | MIKIATRKYLGKQNVYGIGVERDHNFALKNGFIAHNMFNGTVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLEHHHHHH |
| SEQ ID NO: 18 | S_{04b}- Npu_{N}^{C1X,C-S}-G₄S-Cys- Hi_{S6} | Variant derived from SEQ ID NO: 4; constructed by adding linker, immobilization moiety, and purification tag fusion partners at the C-terminus of the N-Intein Ligand. | MGDGHGXLSYETEILTVEYGLLPIGKIVEKRIESTVYSVDNNGNIYTQPVAQWHDRGEQEVFEYSLEDGSLIRATKDHKFMTVDGQMLPIDEIFERELDLMRVDNLPNGGGGSCHHHHHH |

Note: by convention, residue numbering of INT_{C} segment excludes the formylmethionine translation of the start codon, then resumes numbering from the last residue of the INT_{N} segment.

In one example, the solid support substrate can be a solid chromatographic resin backbone, such as a crosslinked agarose. It can also be a membrane, a monolith, or magnetic beads. The term “solid support matrix” or “solid matrix” refers to the solid backbone material of the resin which material contains reactive functionality permitting the covalent attachment of ligand (such as N-Intein Ligand) thereto. The backbone material can be inorganic (e.g., silica) or organic. When the backbone material is organic, it is preferably a solid polymer and suitable organic polymers are well known in the art. Solid support matrices suitable for use in the resins described herein include, by way of example, cellulose, regenerated cellulose, agarose, silica, coated silica, dextran, polymers (such as polyacrylates, polystyrene, polyacrylamide, polymethacrylamide including commercially available polymers such as Fractogel, Enzacryl, and Azlactone), copolymers (such as copolymers of styrene and divinyl- benzene), mixtures thereof and the like. Also, co-, ter- and higher polymers can be used provided that at least one of the monomers contains or can be derivatized to contain a reactive functionality in the resulting polymer. In an additional embodiment, the solid support matrix can contain ionizable functionality incorporated into the backbone thereof.

Reactive functionalities of the solid support matrix substrate, permitting covalent attachment of the N-Intein ligand are well known in the art. Such functionalities react with specific peptide moieties including hydroxyl, carboxyl, thiol, amino, and the like. Conventional chemistry permits use of these functional groups to covalently attach ligands, such as N-Intein Ligands, thereto. Additionally, conventional chemistry permits the inclusion of such groups on the solid support matrix. For example, carboxy groups can be incorporated directly by employing acrylic acid or an ester thereof in the polymerization process. Upon polymerization, carboxyl groups are present if acrylic acid is employed or the polymer can be derivatized to contain carboxyl groups if an acrylate ester is employed.

Affinity tags can be peptide or protein sequences expressed in fusion to the N- or C-terminus of proteins, which confers specific chemical or physical properties that can aid in purifying the protein from cells. Cells expressing a peptide comprising an affinity tag can be pelleted, lysed, and the cell lysate applied to a column, resin or other solid support that displays a ligand to the affinity tags. The affinity tag and any fused peptides are bound to the solid support, which can also be washed several times with buffer to eliminate unbound (contaminant) proteins. A protein of interest, if attached to an affinity tag, can be eluted from the solid support via a buffer that causes the affinity tag to dissociate from the ligand resulting in a purified protein, or can be cleaved from the bound affinity tag using a soluble protease

Examples of affinity tags can be found in Kimple et al. Curr Protoc Protein Sci 2004 Sep; Arnau et al. Protein Expr Purif 2006 Jul; 48(1) 1-13; Azarkan et al. J Chromatogr B Analyt Technol Biomed Life Sci 2007 Apr 15; 849(1-2) 81-90; and Waugh et al. Trends Biotechnol 2005 Jun; 23(6) 316-20, all hereby incorporated by reference in their entirety for their teaching of examples of affinity tags.

Affinity tags can also be used to facilitate the purification of a protein of interest using the disclosed modified peptides through a variety of methods, including, but not limited to, selective precipitation, ion exchange chromatography, binding to precipitation-capable ligands, dialysis (by changing the size and/or charge of the target protein) and other highly selective separation methods.

The N-Intein Ligand can further comprise a sensitivity-enhancing motif (SEM), which renders the splicing or cleaving activity of the assembled intein complex highly sensitive to extrinsic conditions. This sensitivity-enhancing motif can render a cleaving-active intein complex (an N-Intein Ligand bound with an INT_{C}-tagged protein of interest) more likely to cleave under certain conditions. Therefore, the sensitivity-enhancing motif can render the split intein more sensitive to extrinsic conditions when compared to a native, or naturally occurring, intein.

A list of inteins is found below in Table 2. All inteins have the potential to be made into split inteins, while some inteins naturally exist in a split form. All of the inteins found in Table 2 either exist as split inteins, or have the potential to be made into split inteins.

TABLE 2

Naturally Occurring Inteins
Eucarya

| | | |
|---|---|---|
| Intein Name | Organism Name | Organism Description |
| APMV Pol | Acanthomoeba polyphaga Mimivirus | isolate=“Rowbotham-Bradford″, Virus, infects Amoebae, taxon:212035 |
| Abr PRP8 | Aspergillus brevipes FRR2439 | Fungi, ATCC 16899, taxon:75551 |
| Aca-G186AR PRP8 | Ajellomyces capsulatus G186AR | Taxon:447093, strain G186AR |
| Aca-H143 PRP8 | Ajellomyces capsulatus H143 | Taxon:544712 |
| Aca-JER2004 PRP8 | Ajellomyces capsulatus (anamorph: Histoplasma capsulatum) | strain=JER2004, taxon:5037, Fungi |
| Aca-NAm1 PRP8 | Ajellomyces capsulatus NAm1 | strain”NAm1″, taxon:339724 |
| Ade-ER3 PRP8 | Ajellomyces dermatitidis ER-3 | Human fungal pathogen.taxon:559297 |
| Ade-SLH14081 PRP8 | Ajellomyces dermatitidis SLH14081 | Human fungal pathogen |
| Afu-Af293 PRP8 | Aspergillus fumigatus var. ellipticus, strain Af293 | Human pathogenic fungus, taxon:330879 |
| Afu-FRR0163 PRP8 | Aspergillus fumigatus strain FRR0163 | Human pathogenic fungus, taxon:5085 |
| Afu-NRRL5109 PRP8 | Aspergillus fumigatus var. ellipticus, strain NRRL 5109 | Human pathogenic fungus, taxon:41121 |
| Agi-NRRL6136 PRP8 | Aspergillus giganteus Strain NRRL 6136 | Fungus, taxon:5060 |
| Ani-FGSCA4 PRP8 | Aspergillus nidulans FGSC A | Filamentous fungus, taxon:227321 |
| Avi PRP8 | Aspergillus viridinutans strain FRR0577 | Fungi, ATCC 16902, taxon:75553 |
| Bci PRP8 | Botrytis cinerea (teleomorph of Botryotinia fuckeliana B05.10) | Plant fungal pathogen |
| Bde-JEL197 RPB2 | Batrachochytrium dendrobatidis JEL197 | Chytrid fungus, isolate=”AFTOL-ID 21″, taxon: 109871 |
| Bde-JEL423 PRP8-1 | Batrachochytrium dendrobatidis JEL423 | Chytrid fungus, isolate JEL423, taxon 403673 |
| Bde-JEL423 PRP8-2 | Batrachochytrium dendrobatidis JEL423 | Chytrid fungus, isolate JEL423, taxon 403673 |
| Bde-JEL423 RPC2 | Batrachochytrium dendrobatidis JEL423 | Chytrid fungus, isolate JEL423, taxon 403673 |
| Bde-JEL423 eIF-5B | Batrachochytrium dendrobatidis JEL423 | Chytrid fungus, isolate JEL423, taxon 403673 |
| Bfu-B05 PRP8 | Botryotinia fuckeliana B05.10 | Taxon:332648 |
| CIV RIR1 | Chilo iridescent virus | dsDNA eucaryotic virus, taxon: 10488 |
| CV-NY2A ORF212392 | Chlorella virus NY2A infects Chlorella NC64A, which infects Paramecium bursaria | dsDNA eucaryotic virus,taxon:46021, Family Phycodnaviridae |
| CV-NY2A RIR1 | Chlorella virus NY2A infects Chlorella NC64A, which infects Paramecium bursaria | dsDNA eucaryotic virus,taxon:46021, Family Phycodnaviridae |
| CZIV RIR1 | Costelytra zealandica iridescent virus | dsDNA eucaryotic virus, Taxon:68348 |
| Cba-WM02.98 PRP8 | Cryptococcus bacillisporus strain WM02.98 (aka Cryptococcus neoformans gattii) | Yeast, human pathogen, taxon:37769 |
| Cba-WM728 PRP8 | Cryptococcus bacillisporus strain WM728 | Yeast, human pathogen, taxon:37769 |
| Ceu ClpP | Chlamydomonas eugametos (chloroplast) | Green alga, taxon:3053 |
| Cga PRP8 | Cryptococcus gattii (aka Cryptococcus bacillisporus) | Yeast, human pathogen |
| Cgl VMA | Candida glabrata | Yeast, taxon:5478 |
| Cla PRP8 | Cryptococcus laurentii strain CBS139 | Fungi, Basidiomycete yeast, taxon:5418 |
| Cmo ClpP | Chlamydomonas moewusii, strain UTEX 97 | Green alga, chloroplast gene, taxon:3054 |
| Cmo RPB2 (RpoBb) | Chlamydomonas moewusii, strain UTEX 97 | Green alga, chloroplast gene, taxon:3054 |
| Cne-A PRP8 (Fne-A PRP8) | Filobasidiella neoformans (Cryptococcus neoformans) Serotype A, PHLS_8104 | Yeast, human pathogen |
| Cne-AD PRP8 (Fne-AD PRP8) | Cryptococcus neoformans (Filobasidiella neoformans), Serotype AD, CBS132). | Yeast, human pathogen, ATCC32045, taxon:5207 |
| Cne-JEC21 PRP8 | Cryptococcus neoformans var. neoformans JEC21 | Yeast, human pathogen, serotype=“D” taxon:214684 |
| Cpa ThrRS | Candida parapsilosis, strain CLIB214 | Yeast, Fungus, taxon:5480 |
| Cre RPB2 | Chlamydomonas reinhardtii (nucleus) | Green algae, taxon:3055 |
| CroV Pol | Cafeteria roenbergensis virus BV-PW1 | taxon:693272, Giant virus infecting marine heterotrophic nanoflagellate |
| CroV RIR1 | Cafeteria roenbergensis virus BV-PW1 | taxon:693272, Giant virus infecting marine heterotrophic nanoflagellate |
| CroV RPB2 | Cafeteria roenbergensis virus BV-PW1 | taxon:693272, Giant virus infecting marine heterotrophic nanoflagellate |
| CroV Top2 | Cafeteria roenbergensis virus BV-PW1 | taxon:693272, Giant virus infecting marine heterotrophic nanoflagellate |
| Cst RPB2 | Coelomomyces stegomyiae | Chytrid fungus, isolate=“AFTOL-ID 18”, taxon: 143960 |
| Ctr ThrRS | Candida tropicalis ATCC750 | Yeast |
| Ctr VMA | Candida tropicalis (nucleus) | Yeast |
| Ctr-MYA3404 VMA | Candida tropicalis MYA-3404 | Taxon:294747 |
| Ddi RPC2 | Dictyostelium discoideum strain AX4 (nucleus) | Mycetozoa (a social amoeba) |
| Dhan GLT1 | Debaryomyces hansenii CBS767 | Fungi, Anamorph: Candida famata, taxon:4959 |
| Dhan VMA | Debaryomyces hansenii CBS767 | Fungi, taxon:284592 |
| Eni PRP8 | Emericella nidulans R20 (anamorph: Aspergillus nidulans) | taxon: 162425 |
| Eni-FGSCA4 PRP8 | Emericella nidulans (anamorph: Aspergillus nidulans) FGSC A4 | Filamentous fungus, taxon: 162425 |
| Fte RPB2 (RpoB) | Floydiella terrestris, strain UTEX 1709 | Green alga, chloroplast gene, taxon:51328 |
| Gth DnaB | Guillardia theta (plastid) | Cryptophyte Algae |
| HaV01 Pol | Heterosigma akashiwo virus 01 | Algal virus, taxon:97195, strain HaV01 |
| Hca PRP8 | Histoplasma capsulatum (anamorph: Ajellomyces capsulatus) | Fungi, human pathogen |
| IIV6 RIR1 | Invertebrate iridescent virus 6 | dsDNA eucaryotic virus,taxon: 176652 |
| Kex-CBS379 VMA | Kazachstania exigua, formerly Saccharomyces exiguus, strain CBS379 | Yeast, taxon:34358 |
| Kla-CBS683 VMA | Kluyveromyces lactis, strain CBS683 | Yeast, taxon:28985 |
| Kla-IFO1267 VMA | Kluyveromyces lactis IF01267 | Fungi, taxon:28985 |
| Kla-NRRLY1140 VMA | Kluyveromyces lactis NRRL Y-1140 | Fungi, taxon:284590 |
| Lel VMA | Lodderomyces elongisporus | Yeast |
| Mca-CBS113480 PRP8 | Microsporum canis CBS 113480 | Taxon:554155 |
| Nau PRP8 | Neosartorya aurata NRRL 4378 | Fungus, taxon:41051 |
| Nfe-NRRL5534 PRP8 | Neosartorya fennelliae NRRL 5534 | Fungus, taxon:41048 |
| Nfi PRP8 | Neosartorya fischeri | Fungi |
| Ngl-FR2163 PRP8 | Neosartorya glabra FRR2163 | Fungi, ATCC 16909, taxon:41049 |
| Ngl-FRR1833 PRP8 | Neosartorya glabra FRR1833 | Fungi, taxon:41049, (preliminary identification) |
| Nqu PRP8 | Neosartorya quadricincta, strain NRRL 4175 | taxon:41053 |
| Nspi PRP8 | Neosartorya spinosa FRR4595 | Fungi, taxon:36631 |
| Pabr-Pb01 PRP8 | Paracoccidioides brasiliensis Pb01 | Taxon:502779 |
| Pabr-Pb03 PRP8 | Paracoccidioides brasiliensis Pb03 | Taxon:482561 |
| Pan CHS2 | Podospora anserina | Fungi, Taxon 5145 |
| Pan GLT1 | Podospora anserina | Fungi, Taxon 5145 |
| Pbl PRP8-a | Phycomyces blakesleeanus | Zygomycete fungus, strain NRRL155 |
| Pbl PRP8-b | Phycomyces blakesleeanus | Zygomycete fungus, strain NRRL 155 |
| Pbr-Pb 18 PRP8 | Paracoccidioides brasiliensis Pb18 | Fungi, taxon:121759 |
| Pch PRP8 | Penicillium chrysogenum | Fungus, taxon:5076 |
| Pex PRP8 | Penicillium expansum | Fungus, taxon27334 |
| Pgu GLT 1 | Pichia (Candida) guilliermondii | Fungi, Taxon 294746 |
| Pgu-alt GLT1 | Pichia (Candida) guilliermondii | Fungi |
| Pno GLT1 | Phaeosphaeria nodorum SN15 | Fungi,taxon:321614 |
| Pno RPA2 | Phaeosphaeria nodorum SN15 | Fungi,taxon:321614 |
| Ppu DnaB | Porphyra purpurea (chloroplast) | Red Alga |
| Pst VMA | Pichia stipitis CBS 6054, taxon:322104 | Yeast |
| Ptr PRP8 | Pyrenophora tritici-repentis Pt-1C-BF | Ascomycete fungus, taxon:426418 |
| Pvu PRP8 | Penicillium vulpinum (formerly P. claviforme) | Fungus |
| Pye DnaB | Porphyra yezoensis chloroplast, cultivar U-51 | Red alga, organelle=“plastid:chloroplast”, “taxon:2788 |
| Sas RPB2 | Spiromyces aspiralis NRRL 22631 | Zygomycete fungus, isolate=“AFTOL-ID 185”,taxon:68401 |
| Sca-CBS4309 VMA | Saccharomyces castellii, strain CBS4309 | Yeast, taxon:27288 |
| Sca-IFO1992 VMA | Saccharomyces castellii, strain IFO1992 | Yeast, taxon:27288 |
| Scar VMA | Saccharomyces cariocanus, strain=“UFRJ 50791 | Yeast, taxon: 114526 |
| Sce VMA | Saccharomyces cerevisiae (nucleus) | Yeast, also in Sce strains OUT7163, OUT7045, OUT7163, IFO1992 |
| Sce-DH1-1A VMA | Saccharomyces cerevisiae strain DH1-1A | Yeast, taxon:173900,also in Sce strains OUT7900,OUT7903,OUT711 2 |
| Sce-JAY291 VMA | Saccharomyces cerevisiae JAY291 | Taxon:574961 |
| Sce-OUT7091 VMA | Saccharomyces cerevisiae OUT7091 | Yeast, taxon:4932,also in Sce strains OUT7043, OUT7064 |
| Sce-OUT7112 VMA | Saccharomyces cerevisiae OUT7112 | Yeast, taxon:4932, also in Sce strains OUT7900, OUT7903 |
| Sce-YJM789 VMA | Saccharomyces cerevisiae strain YJM789 | Yeast, taxon:307796 |
| Sda VMA | Saccharomyces dairenensis, strain CBS 421 | Yeast, taxon:27289, Also in Sda strain IFO0211 |
| Sex-IFO1128 VMA | Saccharomyces exiguus, strain=“IFO1128″ | Yeast, taxon:34358 |
| She RPB2 (RpoB) | Stigeoclonium helveticum, strain UTEX 441 | Green alga, chloroplast gene, taxon:55999 |
| Sja VMA | Schizosaccharomyces japonicus yFS275 | Ascomycete fungus, taxon:402676 |
| Spa VMA | Saccharomyces pastorianus IFO11023 | Yeast, taxon:27292 |
| Spu PRP8 | Spizellomyces punctatus | Chytrid fungus, |
| Sun VMA | Saccharomyces unisporus, strain CBS 398 | Yeast, taxon:27294 |
| Tgl VMA | Torulaspora globosa, strain CBS 764 | Yeast, taxon:48254 |
| Tpr VMA | Torulaspora pretoriensis, strain CBS 5080 | Yeast, taxon:35629 |
| Ure-1704 PRP8 | Uncinocarpus reesii | Filamentous fungus |
| Vpo VMA | Vanderwaltozyma polyspora, formerly Kluyveromyces polysporus, strain CBS 2163 | Yeast, taxon:36033 |
| WIV RIR1 | Wiseana iridescent virus | dsDNA eucaryotic virus,taxon:68347 |
| Zba VMA | Zygosaccharomyces bailii, strain CBS 685 | Yeast, taxon:4954 |
| Zbi VMA | Zygosaccharomyces bisporus, strain CBS 702 | Yeast, taxon:4957 |
| Zro VMA | Zygosaccharomyces rouxii, strain CBS 688 | Yeast, taxon:4956 |
| AP-APSE1 dpol | Acyrthosiphon pisum secondary endosymbiot phage 1 | Bacteriophage, taxon:67571 |
| AP-APSE2 dpol | Bacteriophage APSE-2, isolate=T5A | Bacteriophage of Candidatus Hamiltonella defensa, endosymbiot of Acyrthosiphon pisum ,taxon:340054 |
| AP-APSE4 dpol | Bacteriophage of Candidatus Hamiltonella defensa strain 5ATac, endosymbiot of Acyrthosiphon pisum | Bacteriophage, taxon: 568990 |
| AP-APSE5 dpol | Bacteriophage APSE-5 | Bacteriophage of Candidatus Hamiltonella defensa, endosymbiot of Uroleucon rudbeckiae, taxon:568991 |
| AP-Aaphi23 MupF | Bacteriophage Aaphi23, Haemophilus phage Aaphi23 | Actinobacillus actinomycetemcomitans Bacteriophage, taxon:230158 |
| Aae RIR2 | Aquifex aeolicus strain VF5 | Thermophilic chemolithoautotroph, taxon:63363 |
| Aave-AAC001 Aave1721 | Acidovorax avenae subsp. citrulli AAC00-1 | taxon:397945 |
| Aave-AAC001 RIR1 | Acidovorax avenae subsp. citrulli AAC00-1 | taxon:397945 |
| Aave-ATCC 19860 RIR1 | Acidovorax avenae subsp. avenae ATCC 19860 | Taxon:643561 |
| Aba Hyp-02185 | Acinetobacter baumannii ACICU | taxon:405416 |
| Ace RIR1 | Acidothermus cellulolyticus 11B | taxon:351607 |
| Aeh DnaB-1 | Alkalilimnicola ehrlichei MLHE-1 | taxon: 187272 |
| Aeh DnaB-2 | Alkalilimnicola ehrlichei MLHE-1 | taxon: 187272 |
| Aeh RIR1 | Alkalilimnicola ehrlichei MLHE-1 | taxon: 187272 |
| AgP-S1249 MupF | Aggregatibacter phage S1249 | Taxon:683735 |
| Aha DnaE-c | Aphanothece halophytica | Cyanobacterium, taxon:72020 |
| Aha DnaE-n | Aphanothece halophytica | Cyanobacterium, taxon:72020 |
| Alvi-DSM180 GyrA | Allochromatium vinosum DSM 180 | Taxon: 572477 |
| Ama MADE823 | phage uncharacterized protein [Alteromonas macleodii ‘Deep ecotype’] | Probably prophage gene, taxon:314275 |
| Amax-CS328 DnaX | Arthrospira maxima CS-328 | Taxon:513049 |
| Aov DnaE-c | Aphanizomenon ovalisporum | Cyanobacterium, taxon:75695 |
| Aov DnaE-n | Aphanizomenon ovalisporum | Cyanobacterium, taxon:75695 |
| Apl-C1 DnaX | Arthrospira platensis | Taxon:118562, strain C1 |
| Arsp-FB24 DnaB | Arthrobacter species FB24 | taxon:290399 |
| Asp DnaE-c | Anabaena species PCC7120, (Nostoc sp. PCC7120) | Cyanobacterium, Nitrogen-fixing, taxon: 103690 |
| Asp DnaE-n | Anabaena species PCC7120, (Nostoc sp. PCC7120) | Cyanobacterium, Nitrogen-fixing, taxon:103690 |
| Ava DnaE-c | Anabaena variabilis ATCC29413 | Cyanobacterium, taxon:240292 |
| Ava DnaE-n | Anabaena variabilis ATCC29413 | Cyanobacterium, taxon:240292 |
| Avin RIR1 BIL | Azotobacter vinelandii | taxon:354 |
| Bce-MCO3 DnaB | Burkholderia cenocepacia MC0-3 | taxon:406425 |
| Bce-PC184 DnaB | Burkholderia cenocepacia PC184 | taxon:350702 |
| Bse-MLS10 TerA | Bacillus selenitireducens MLS10 | Probably prophage gene, Taxon:439292 |
| BsuP-M1918 RIR1 | B.subtilis M1918 (prophage) | Prophage in B.subtilis M1918. taxon: 157928 |
| BsuP-SPBc2 RIR1 | B.subtilis strain 168 Sp beta c2 prophage | B.subtilis taxon 1423. SPbeta c2 phage, taxon:66797 |
| Bvi IcmO | Burkholderia vietnamiensis G4 | plasmid=“pBVIE03”. taxon:269482 |
| CP-P1201 Thy1 | Corynebacterium phage P1201 | lytic bacter“iophage P1201 from Corynebacterium glutamicum NCHU 87078.Viruses; dsDNA viruses, taxon:384848 |
| Cag RIR1 | Chlorochromatium aggregatum | Motile, phototrophic consortia |
| Cau SpoVR | Chloroflexus aurantiacus J-10-fl | Anoxygenic phototroph,taxon:324602 |
| CbP-C-St RNR | Clostridium botulinum phage C-St | Phage,specific_host=“Clostrid ium botulinum type C strain C-Stockholm, taxon: 12336 |
| CbP-D1873 RNR | Clostridium botulinum phage D | Ssp. phage from Clostridium botulinum type D strain, 1873, taxon:29342 |
| Cbu-Dugway DnaB | Coxiella burnetii Dugway 5J108-111 | Proteobacteria; Legionellales; taxon:434922 |
| Cbu-Goat DnaB | Coxiella burnetii ‘MSU Goat Q177’ | Proteobacteria; Legionellales; taxon:360116 |
| Cbu-RSA334 DnaB | Coxiella burnetii RSA 334 | Proteobacteria; Legionellales; taxon:360117 |
| Cbu-RSA493 DnaB | Coxiella burnetii RSA 493 | Proteobacteria; Legionellales; taxon:227377 |
| Cce Hyp1-Csp-2 | Cyanothece sp. ATCC 51142 | Marine unicellular diazotrophic cyanobacterium, taxon:43989 |
| Cch RIR1 | Chlorobium chlorochromatii CaD3 | taxon:340177 |
| Ccy Hyp1-Csp-1 | Cyanothece sp. CCY0110 | Cyanobacterium, taxon:391612 |
| Ccy Hyp1-Csp-2 | Cyanothece sp. CCY0110 | Cyanobacterium, taxon:391612 |
| Cfl-DSM20109 DnaB | Cellulomonas flavigena DSM 20109 | Taxon:446466 |
| Chy RIR1 | Carboxydothermus hydrogenoformans Z-2901 | Thermophile, taxon=246194 |
| Ckl PTerm | Clostridium kluyveri DSM 555 | plasmid=“pCKL555A”, taxon:431943 |
| Cra-CS505 DnaE-c | Cylindrospermopsis raciborskii CS-505 | Taxon:533240 |
| Cra-CS505 DnaE-n | Cylindrospermopsis raciborskii CS-505 | Taxon:533240 |
| Cra-CS505 GyrB | Cylindrospermopsis raciborskii CS-505 | Taxon:533240 |
| Csp-CCY0110 DnaE-c | Cyanothece sp. CCY0110 | Taxon:391612 |
| Csp-CCY0110 DnaE-n | Cyanothece sp. CCY0110 | Taxon:391612 |
| Csp-PCC7424 DnaE-c | Cyanothece sp. PCC 7424 | Cyanobacterium, taxon:65393 |
| Csp-PCC7424 DnaE-n | Cyanothece sp. PCC7424 | Cyanobacterium, taxon:65393 |
| Csp-PCC7425 DnaB | Cyanothece sp. PCC 7425 | Taxon:395961 |
| Csp-PCC7822 DnaE-n | Cyanothece sp. PCC 7822 | Taxon:497965 |
| Csp-PCC8801 DnaE-c | Cyanothece sp. PCC 8801 | Taxon:41431 |
| Csp-PCC8801 DnaE-n | Cyanothece sp. PCC 8801 | Taxon:41431 |
| Cth ATPase BIL | Clostridium thermocellum | ATCC27405, taxon:203119 |
| Cth-ATCC27405 TerA | Clostridium thermocellum ATCC27405 | Probable prophage, ATCC27405, taxon:203119 |
| Cth-DSM2360 TerA | Clostridium thermocellum DSM 2360 | Probably prophage gene,Taxon:572545 |
| Cwa DnaB | Crocosphaera watsonii WH 8501 (Synechocystis sp. WH 8501) | taxon: 165597 |
| Cwa DnaE-c | Crocosphaera watsonii WH 8501 (Synechocystis sp. WH 8501) | Cyanobacterium, taxon: 165597 |
| Cwa DnaE-n | Crocosphaera watsonii WH 8501 (Synechocystis sp. WH 8501) | Cyanobacterium, taxon: 165597 |
| Cwa PEP | Crocosphaera watsonii WH 8501 (Synechocystis sp. WH 8501) | taxon: 165597 |
| Cwa RIR1 | Crocosphaera watsonii WH 8501 (Synechocystis sp. WH 8501) | taxon: 165597 |
| Daud RIR1 | Candidatus Desulforudis audaxviator MP 104C | taxon:477974 |
| Dge DnaB | Deinococcus geothermalis DSM11300 | Thermophilic, radiation resistant |
| Dha-DCB2 RIR1 | Desulfitobacterium hafniense DCB-2 | Anaerobic dehalogenating bacteria, taxon:49338 |
| Dha-Y51 RIR1 | Desulfitobacterium hafniense Y51 | Anaerobic dehalogenating bacteria, taxon: 138119 |
| Dpr-MLMS1 RIR1 | delta proteobacterium MLMS-1 | Taxon:262489 |
| Dra RIR1 | Deinococcus radiodurans R1,TIGR strain | Radiation resistant, taxon: 1299 |
| Dra Snf2-c | Deinococcus radiodurans R1, TIGR strain | Radiation and DNA damage resistent, taxon:1299 |
| Dra Snf2-n | Deinococcus radiodurans R1, TIGR strain | Radiation and DNA damage resistent, taxon:1299 |
| Dra-ATCC13939 Snf2 | Deinococcus radiodurans R1, ATCC13939/Brooks & Murray strain | Radiation and DNA damage resistent, taxon:1299 |
| Dth UDP GD | Dictyoglomus thermophilum H-6-12 | strain=“H-6-12; ATCC 35947, taxon:309799 |
| Dvul ParB | Desulfovibrio vulgaris subsp. vulgaris DP4 | taxon:391774 |
| EP-Min27 Primase | Enterobacteria phage Min27 | bacteriphage of host=“Escherichia coli O157:H7 str. Min27” |
| Fal DnaB | Frankia alni ACN14a | Plant symbiot, taxon:326424 |
| Fsp-CcI3 RIR1 | Frankia species CcI3 | taxon: 106370 |
| Gob DnaE | Gemmata obscuriglobus UQM2246 | Taxon 114, TIGR genome strain, budding bacteria |
| Gob Hyp | Gemmata obscuriglobus UQM2246 | Taxon 114, TIGR genome strain, budding bacteria |
| Gvi DnaB | Gloeobacter violaceus, PCC 7421 | taxon:33072 |
| Gvi RIR1-1 | Gloeobacter violaceus, PCC 7421 | taxon:33072 |
| Gvi RIR1-2 | Gloeobacter violaceus, PCC 7421 | taxon:33072 |
| Hhal DnaB | Halorhodospira halophila SL1 | taxon:349124 |
| Kfl-DSM17836 DnaB | Kribbella flavida DSM 17836 | Taxon:47943 5 |
| Kra DnaB | Kineococcus radiotolerans SRS30216 | Radiation resistant |
| LLP-KSY1 PolA | Lactococcus phage KSY1 | Bacteriophage, taxon:388452 |
| LP-phiHSIC Helicase | Listonella pelagia phage phiHSIC | taxon:310539,a pseudotemperate marine phage of Listonella pelagia |
| Lsp-PCC8106 GyrB | Lyngbya sp. PCC 8106 | Taxon:313612 |
| MP-Be DnaB | Mycobacteriophage Bethlehem | Bacteriophage, taxon:260121 |
| MP-Be gp51 | Mycobacteriophage Bethlehem | Bacteriophage, taxon:260121 |
| MP-Catera gp206 | Mycobacteriophage Catera | Mycobacteriophage, taxon:373404 |
| MP-KBG gp53 | Mycobacterium phage KBG | Taxon:540066 |
| MP-Mcjwl DnaB | Mycobacteriophage CJW1 | B acteriophage, taxon: 205869 |
| MP-Omega DnaB | Mycobacteriophage Omega | Bacteriophage, taxon:205879 |
| MP-U2 gp50 | Mycobacteriophage U2 | Bacteriophage, taxon:260120 |
| Maer-NIES843 DnaB | Microcystis aeruginosa NIES-843 | Bloom-forming toxic cyanobacterium,taxon:449447 |
| Maer-NIES843 DnaE-c | Microcystis aeruginosa NIES-843 | Bloom-forming toxic cyanobacterium,taxon:449447 |
| Maer-NIES843 DnaE-n | Microcystis aeruginosa NIES-843 | Bloom-forming toxic cyanobacterium,taxon:449447 |
| Mau-ATCC27029 GyrA | Micromonospora aurantiaca ATCC 27029 | Taxon:644283 |
| Mav-104 DnaB | Mycobacterium avium 104 | taxon:243243 |
| Mav-ATCC25291 DnaB | Mycobacterium avium subsp. avium ATCC 25291 | Taxon:553481 |
| Mav-ATCC35712 DnaB | Mycobacterium avium | ATCC35712, taxon 1764 |
| Mav-PT DnaB | Mycobacterium avium subsp. paratuberculosis str. k10 | taxon:262316 |
| Mbo Pps1 | Mycobacterium bovis subsp. bovis AF2122/97 | strain=“AF2122/97”, taxon:233413 |
| Mbo RecA | Mycobacterium bovis subsp. bovis AF2122/97 | taxon:233413 |
| Mbo SufB (Mbo Pps1) | Mycobacterium bovis subsp. bovis AF2122/97 | taxon:233413 |
| Mbo-1173P DnaB | Mycobacterium bovis BCG Pasteur 1173P | strain= BCG Pasteur 1173P2,,taxon:410289 |
| Mbo-AF2122 DnaB | Mycobacterium bovis subsp. bovis AF2122/97 | strain=“AF2122/97”, taxon:233413 |
| Mca MupF | Methylococcus capsulatus Bath, prophage MuMc02 | prophage MuMc02, taxon:243233 |
| Mca RIR1 | Methylococcus capsulatus Bath | taxon:243233 |
| Mch RecA | Mycobacterium chitae | IP14116003, taxon: 1792 |
| Mcht-PCC7420 DnaE-1 | Microcoleus chthonoplastes PCC7420 | Cyanobacterium, taxon:118168 |
| Mcht-PCC7420 DnaE-2c | Microcoleus chthonoplastes PCC7420 | Cyanobacterium, taxon:118168 |
| Mcht-PCC7420 DnaE-2n | Microcoleus chthonoplastes PCC7420 | Cyanobacterium, taxon:118168 |
| Mcht-PCC7420 GyrB | Microcoleus chthonoplastes PCC 7420 | Taxon:118168 |
| Mcht-PCC7420 RIR1-1 | Microcoleus chthonoplastes PCC 7420 | Taxon:118168 |
| Mcht-PCC7420 RIR1-2 | Microcoleus chthonoplastes PCC 7420 | Taxon:118168 |
| Mex Helicase | Methylobacterium extorquens AM1 | Alphaproteob acteri a |
| Mex TrbC | Methylobacterium extorquens AM1 | Alphaproteobacteria |
| Mfa RecA | Mycobacterium fallax | CITP8139, taxon:1793 |
| Mfl GyrA | Mycobacterium flavescens Fla0 | taxon:1776, reference #930991 |
| Mfl RecA | Mycobacterium flavescens Fla0 | strain=Fla0, taxon:1776, ref. #930991 |
| Mfl-ATCC14474 RecA | Mycobacterium flavescens, ATCC14474 | strain=ATCC14474,taxon: 177 6, ref #930991 |
| Mfl-PYR-GCK DnaB | Mycobacterium flavescens PYR-GCK | taxon:350054 |
| Mga GyrA | Mycobacterium gastri | HP4389, taxon:1777 |
| Mga RecA | Mycobacterium gastri | HP4389, taxon:1777 |
| Mga SufB (Mga Pps1) | Mycobacterium gastri | HP4389, taxon:1777 |
| Mgi-PYR-GCK DnaB | Mycobacterium gilvum PYR-GCK | taxon:350054 |
| Mgi-PYR-GCK GyrA | Mycobacterium gilvum PYR-GCK | taxon:350054 |
| Mgo GyrA | Mycobacterium gordonae | taxon:1778, reference number 930835 |
| Min-1442 DnaB | Mycobacterium intracellulare | strain 1442, taxon:1767 |
| Min-ATCC13950 GyrA | Mycobacterium intracellulare ATCC 13950 | Taxon:487521 |
| Mkas GyrA | Mycobacterium kansasii | taxon: 1768 |
| Mkas-ATCC 12478 GyrA | Mycobacterium kansasii ATCC 12478 | Taxon:557599 |
| Mle-Br4923 GyrA | Mycobacterium leprae Br4923 | Taxon:561304 |
| Mle-TN DnaB | Mycobacterium leprae, strain TN | Human pathogen, taxon:1769 |
| Mle-TN GyrA | Mycobacterium leprae TN | Human pathogen, STRAIN=TN, taxon:1769 |
| Mle-TN RecA | Mycobacterium leprae, strain TN | Human pathogen, taxon:1769 |
| Mle-TN SufB (Mle Pps1) | Mycobacterium leprae | Human pathogen, taxon:1769 |
| Mma GyrA | Mycobacterium malmoense | taxon: 1780 |
| Mmag Magn8951 BIL | Magnetospirillum magnetotacticum MS-1 | Gram negative, taxon:272627 |
| Msh RecA | Mycobacterium shimodei | ATCC27962, taxon:29313 |
| Msm DnaB-1 | Mycobacterium smegmatis MC2 155 | MC2 155,taxon:246196 |
| Msm DnaB-2 | Mycobacterium smegmatis MC2 155 | MC2 155,taxon:246196 |
| Msp-KMS DnaB | Mycobacterium species KMS | taxon: 189918 |
| Msp-KMS GyrA | Mycobacterium species KMS | taxon: 189918 |
| Msp-MCS DnaB | Mycobacterium species MCS | taxon: 164756 |
| Msp-MCS GyrA | Mycobacterium species MCS | taxon: 164756 |
| Mthe RecA | Mycobacterium thermoresistibile | ATCC 19527, taxon: 1797 |
| Mtu SufB (Mtu Pps1) | Mycobacterium tuberculosis strains H37Rv & CDC1551 | Human pathogen, taxon:83332 |
| Mtu-C RecA | Mycobacterium tuberculosis C | Taxon:348776 |
| Mtu-CDC1551 DnaB | Mycobacterium tuberculosis, CDC1551 | Human pathogen, taxon:83332 |
| Mtu-CPHL RecA | Mycobacterium tuberculosis CPHL_A | Taxon:611303 |
| Mtu-Canetti RecA | Mycobacterium tuberculosis /strain=“Canetti” | Taxon: 1773 |
| Mtu-EAS054 RecA | Mycobacterium tuberculosis EAS054 | Taxon:520140 |
| Mtu-F 11 DnaB | Mycobacterium tuberculosis, strain F11 | taxon:336982 |
| Mtu-H37Ra DnaB | Mycobacterium tuberculosis H37Ra | ATCC 25177, taxon:419947 |
| Mtu-H37Rv DnaB | Mycobacterium tuberculosis H37Rv | Human pathogen, taxon:83332 |
| Mtu-H37Rv RecA | Mycobacterium tuberculosis H37Rv,Also CDC1551 | Human pathogen, taxon:83332 |
| Mtu-Haarlem DnaB | Mycobacterium tuberculosis str. Haarlem | Taxon:395095 |
| Mtu-K85 RecA | Mycobacterium tuberculosis K85 | Taxon:611304 |
| Mtu-R604 RecA-n | Mycobacterium tuberculosis ‘98-R604 INH-RIF-EM’ | Taxon:555461 |
| Mtu-So93 RecA | Mycobacterium tuberculosis So93/sub_species=“Canetti” | Human pathogen, taxon:1773 |
| Mtu-T17 RecA-c | Mycobacterium tuberculosis T17 | Taxon:537210 |
| Mtu-T17 RecA-n | Mycobacterium tuberculosis T17 | Taxon:537210 |
| Mtu-T46 RecA | Mycobacterium tuberculosis T46 | Taxon:611302 |
| Mtu-T85 RecA | Mycobacterium tuberculosis T85 | Taxon:520141 |
| Mtu-T92 RecA | Mycobacterium tuberculosis T92 | Taxon:515617 |
| Mvan DnaB | Mycobacterium vanbaalenii PYR-1 | taxon:350058 |
| Mvan GyrA | Mycobacterium vanbaalenii PYR-1 | taxon:350058 |
| Mxa RAD25 | Myxococcus xanthus DK1622 | Deltaproteobacteria |
| Mxe GyrA | Mycobacterium xenopi strain IMM5024 | taxon: 1789 |
| Naz-0708 RIR1-1 | Nostoc azollae 0708 | Taxon:551115 |
| Naz-0708 RIR1-2 | Nostoc azollae 0708 | Taxon:551115 |
| Nfa DnaB | Nocardia farcinica IFM 10152 | taxon:247156 |
| Nfa Nfa15250 | Nocardia farcinica IFM 10152 | taxon:247156 |
| Nfa RIR1 | Nocardia farcinica IFM 10152 | taxon:247156 |
| Nosp-CCY9414 DnaE-n | Nodularia spumigena CCY9414 | Taxon:313624 |
| Npu DnaB | Nostoc punctiforme | Cyanobacterium,taxon: 63 73 7 |
| Npu GyrB | Nostoc punctiforme | Cyanobacterium,taxon: 63 73 7 |
| Npu-PCC73102 DnaE-c | Nostoc punctiforme PCC73102 | Cyanobacterium,taxon: 63 73 7, ATCC29133 |
| Npu-PCC73102 DnaE-n | Nostoc punctiforme PCC73102 | Cyanobacterium,taxon: 63 73 7, ATCC29133 |
| Nsp-JS614 DnaB Nsp-JS614 TOPRIM | Nocardioides species JS614 Nocardioides species JS614 | taxon: 196162 taxon: 196162 |
| Nsp-PCC7120 DnaB | Nostoc species PCC7120, (Anabaena sp. PCC7120) | Cyanobacterium, Nitrogen-fixing, taxon:103690 |
| Nsp-PCC7120 DnaE-c | Nostoc species PCC7120, (Anabaena sp. PCC7120) | Cyanobacterium, Nitrogen-fixing, taxon: 103690 |
| Nsp-PCC7120 DnaE-n | Nostoc species PCC7120, (Anabaena sp. PCC7120) | Cyanobacterium, Nitrogen-fixing, taxon:103690 |
| Nsp-PCC7120 RIR1 | Nostoc species PCC7120, (Anabaena sp. PCC7120) | Cyanobacterium, Nitrogen-fixing, taxon: 103690 |
| Oli DnaE-c | Oscillatoria limnetica str. ‘Solar Lake’ | Cyanobacterium, taxon:262926 |
| Oli DnaE-n | Oscillatoria limnetica str. ‘Solar Lake’ | Cyanobacterium, taxon:262926 |
| PP-PhiEL Helicase | Pseudomonas aeruginosa phage phiEL | Phage infects Pseudomonas aeruginosa, taxon:273133 phage infects Pseudomonas aeruginosa, taxon:273133 |
| PP-PhiEL ORF11 | Pseudomonas aeruginosa phage phiEL | |
| PP-PhiEL ORF39 | Pseudomonas aeruginosa phage phiEL | Phage infects Pseudomonas aeruginosa, taxon:273133 |
| PP-PhiEL ORF40 | Pseudomonas aeruginosa phage phiEL | phage infects Pseudomonas aeruginosa, taxon:273133 |
| Pfl Fha BIL | Pseudomonas fluorescens Pf-5 | Plant commensal organism, taxon:220664 |
| Plut RIR1 | Pelodictyon luteolum DSM 273 | Green sulfur bacteria, Taxon 319225 |
| Pma-EXH1 GyrA | Persephonella marina EX-H1 | Taxon: 123214 |
| Pma-ExH1 DnaE | Persephonella marina EX-H1 | Taxon: 123214 |
| Pna RIR1 | Polaromonas naphthalenivorans CJ2 | taxon:365044 |
| Pnuc DnaB | Polynucleobacter sp. QLW-P1DMWA-1 | taxon:312153 |
| Posp-JS666 DnaB | Polaromonas species JS666 | taxon:296591 |
| Posp-JS666 RIR1 | Polaromonas species JS666 | taxon:296591 |
| Pssp-A1-1 Fha | Pseudomonas species A1-1 | |
| Psy Fha | Pseudomonas syringae pv. tomato str. DC3000 | Plant (tomato) pathogen, taxon:223283 |
| Rbr-D9 GyrB | Raphidiopsis brookii D9 | Taxon:533247 |
| Rce RIR1 | Rhodospirillum centenum SW | taxon:414684,ATCC 51521 |
| Rer-SK121 DnaB | Rhodococcus erythropolis SK121 | Taxon:596309 |
| Rma DnaB | Rhodothermus marinus | Thermophile, taxon: 29549 |
| Rma-DSM4252 DnaB | Rhodothermus marinus DSM 4252 | Taxon:518766 |
| Rma-DSM4252 DnaE | Rhodothermus marinus DSM 4252 | Thermophile, taxon:518766 |
| Rsp RIR1 | Roseovarius species 217 | taxon:314264 |
| SaP-SETP12 dpol | Salmonella phage SETP12 | Phage,taxon:424946 |
| SaP-SETP3 Helicase | Salmonella phage SETP3 | Phage,taxon:424944 |
| SaP-SETP3 dpol | Salmonella phage SETP3 | Phage,taxon:424944 |
| SaP-SETP5 dpol | Salmonella phage SETP5 | Phage,taxon:424945 |
| Sare DnaB | Salinispora arenicola CNS-205 | taxon:391037 |
| Sav RecG Helicase | Streptomyces avermitilis MA-4680 | taxon:227882, ATCC 31267 |
| Sel-PC6301 RIR1 | Synechococcus elongatus PCC 6301 | taxon:269084 Berkely strain 6301~equivalent name: Ssp PCC 6301-synonym: Anacystis nudulans |
| Sel-PC7942 DnaE-c | Synechococcus elongatus PC7942 | taxon:1140 |
| Sel-PC7942 DnaE-n | Synechococcus elongatus PC7942 | taxon:1140 |
| Sel-PC7942 RIR1 | Synechococcus elongatus PC7942 | taxon:1140 |
| Sel-PCC6301 DnaE-c | Synechococcus elongatus PCC 6301 and PCC7942 | Cyanobacterium, taxon:269084,“Berkely strain 6301~equivalent name: Synechococcus sp. PCC 6301~synonym: Anacystis nudulans” |
| Sel-PCC6301 DnaE-n Sep RIR1 | Synechococcus elongatus PCC 6301 Staphylococcus epidermidis RP62A | Cyanobacterium, taxon:269084“Berkely strain 6301~equivalent name: Synechococcus sp. PCC 6301~synonym: Anacystis nudulans” taxon:176279 |
| ShP-Sfv-2a-2457T-n Primase | Shigella flexneri 2a str. 2457T | Putative bacteriphage |
| ShP-Sfv-2a-301-n Primase | Shigella flexneri 2a str. 301 | Putative bacteriphage |
| ShP-Sfv-5 Primase | Shigella flexneri 5 str. 8401 | Bacteriphage,isolation_source _epidemic, taxon:373384 |
| SoP-SO1 dpol | Sodalis phage SO-1 | Phage/isolation_source=“Soda lis glossinidius strain GA-SG, secondary symbiont of Glossina austeni (Newstead)” |
| Spl DnaX | Spirulina platensis, strain C1 | Cyanobacterium, taxon:1156 |
| Sru DnaB | Salinibacter ruber DSM 13855 | taxon:309807,strain=“DSM 13855; M31” |
| Sru PolBc | Salinibacter ruber DSM 13855 | taxon:309807,strain=”DSM 13855; M31” |
| Sru RIR1 | Salinibacter ruber DSM 13855 | taxon:309807,strain=“DSM 13855; M31” |
| Ssp DnaB | Synechocystis species, strain PCC6803 | Cyanobacterium, taxon:1148 |
| Ssp DnaE-c | Synechocystis species, strain PCC6803 | Cyanobacterium, taxon:1148 |
| Ssp DnaE-n | Synechocystis species, strain PCC6803 | Cyanobacterium, taxon:1148 |
| Ssp DnaX | Synechocystis species, strain PCC6803 | Cyanobacterium, taxon:1148 |
| Ssp GyrB | Synechocystis species, strain PCC6803 | Cyanobacterium, taxon:1148 |
| Ssp-JA2 DnaB | Synechococcus species JA-2-3B’a(2- 13) | Cyanobacterium, Taxon:321332 |
| Ssp-JA2 RIR1 | Synechococcus species JA-2-3B’a(2- 13) | Cyanobacterium, Taxon:321332 |
| Ssp-JA3 DnaB | Synechococcus species JA-3-3Ab | Cyanobacterium, Taxon:321327 |
| Ssp-JA3 RIR1 | Synechococcus species JA-3-3Ab | Cyanobacterium, Taxon:321327 |
| Ssp-PCC7002 DnaE-c | Synechocystis species, strain PCC 7002 | Cyanobacterium, taxon: 32049 |
| Ssp-PCC7002 DnaE-n | Synechocystis species, strain PCC 7002 | Cyanobacterium, taxon: 32049 |
| Ssp-PCC7335 RIR1 | Synechococcus sp. PCC 7335 | Taxon:91464 |
| StP-Twort ORF6 | Staphylococcus phage Twort | Phage, taxon 55510 |
| Susp-NBC371 DnaB intein | Sulfurovum sp. NBC37-1 | taxon:387093 |
| Taq-Y51MC23 DnaE | Thermus aquaticus Y51MC23 | Taxon:498848 |
| Taq-Y51MC23 RIR1 | Thermus aquaticus Y51MC23 | Taxon:498848 |
| Tcu-DSM43183 RecA | Thermomonospora curvata DSM 43183 | Taxon:471852 |
| Tel DnaE-c | Thermosynechococcus elongatus BP- 1 | Cyanobacterium, taxon:197221 |
| Tel DnaE-n | Thermosynechococcus elongatus BP- 1 | Cyanobacterium, |
| Ter DnaB-1 | Trichodesmium erythraeum IMS101 | Cyanobacterium, taxon:203124 |
| Ter DnaB-2 | Trichodesmium erythraeum IMS101 | Cyanobacterium, taxon:203124 |
| TerDnaE-1 | Trichodesmium erythraeum IMS101 | Cyanobacterium, taxon:203124 |
| Ter DnaE-2 | Trichodesmium erythraeum IMS101 | Cyanobacterium, taxon:203124 |
| TerDnaE-3c | Trichodesmium erythraeum IMS101 | Cyanobacterium, taxon:203124 |
| TerDnaE-3n | Trichodesmium erythraeum IMS101 | Cyanobacterium, taxon:203124 |
| Ter GyrB | Trichodesmium erythraeum IMS101 | Cyanobacterium, taxon:203124 |
| Ter Ndse-1 | Trichodesmium erythraeum IMS101 | Cyanobacterium, taxon:203124 |
| Ter Ndse-2 | Trichodesmium erythraeum IMS101 | Cyanobacterium, taxon:203124 |
| Ter RIR1-1 | Trichodesmium erythraeum IMS101 | Cyanobacterium, taxon:203124 |
| Ter RIR1-2 | Trichodesmium erythraeum IMS101 | Cyanobacterium, taxon:203124 |
| Ter RIR1-3 | Trichodesmium erythraeum IMS101 | Cyanobacterium, taxon:203124 |
| Ter RIR1-4 | Trichodesmium erythraeum IMS101 | Cyanobacterium, taxon:203124 |
| Ter Snf2 | Trichodesmium erythraeum IMS101 | Cyanobacterium, taxon:203124 |
| Ter ThyX | Trichodesmium erythraeum IMS101 | Cyanobacterium, taxon:203124 |
| Tfus RecA-1 | Thermobifida fusca YX | Thermophile,taxon:269800 |
| Tfus RecA-2 | Thermobifida fusca YX | Thermophile,taxon:269800 |
| Tfus Tfu2914 | Thermobifida fusca YX | Thermophile,taxon:269800 |
| Thsp-K90 RIR1 | Thioalkalivibrio sp. K90mix | Taxon:396595 |
| Tth-DSM571 RIR1 | Thermoanaerobacterium thermosaccharolyticum DSM 571 | Taxon:580327 |
| Tth-HB27 DnaE-1 | Thermus thermophilus HB27 | thermophile, taxon:262724 |
| Tth-HB27 DnaE-2 | Thermus thermophilus HB27 | thermophile, taxon:262724 |
| Tth-HB27 RIR1-1 | Thermus thermophilus HB27 | thermophile, taxon:262724 |
| Tth-HB27 RIR1-2 | Thermus thermophilus HB27 | thermophile, taxon:262724 |
| Tth-HB8 DnaE-1 | Thermus thermophilus HB8 | thermophile, taxon:300852 |
| Tth-HB8 DnaE-2 | Thermus thermophilus HB8 | thermophile, taxon:300852 |
| Tth-HB8 RIR1-1 | Thermus thermophilus HB8 | thermophile, taxon:300852 |
| Tth-HB8 RIR1-2 | Thermus thermophilus HB8 | thermophile, taxon:300852 |
| Tvu DnaE-c | Thermosynechococcus vulcanus | Cyanobacterium, taxon:32053 |
| Tvu DnaE-n | Thermosynechococcus vulcanus | Cyanobacterium, taxon:32053 |
| Tye RNR-1 | Thermodesulfovibrio yellowstonii DSM 11347 | taxon:289376 |
| Tye RNR-2 | Thermodesulfovibrio yellowstonii DSM 11347 | taxon:289376 |
| Ape APE0745 | Aeropyrum pemix K1 | Thermophile, taxon:56636 |
| Cme-boo Pol-II | Candidatus Methanoregula boonei 6A8 | taxon:456442 |
| Fac-Fer1 RIR1 | Ferroplasma acidarmanus, taxon:97393 and taxon 261390 | strain Fer1, eats iron |
| Fac-Fer1 SufB (Fac Pps1) | Ferroplasma acidarmanus | strain fer1, eats iron,taxon:97393 |
| Fac-TypeI RIR1 | Ferroplasma acidarmanus type I, | Eats iron, taxon 261390 |
| Fac-typeI SufB (Fac Pps1) | Ferroplasma acidarmanus | Eats iron,taxon:261390 |
| HmaCDC21 | Haloarcula marismortui ATCC 43049 | taxon:272569, |
| Hma Pol-II | Haloarcula marismortui ATCC 43049 | taxon:272569, |
| Hma PolB | Haloarcula marismortui ATCC 43049 | taxon:272569, |
| Hma TopA | Haloarcula marismortui ATCC 43049 | taxon:272569 |
| Hmu-DSM12286 MCM | Halomicrobium mukohataei DSM 12286 | taxon: 485914 (Halobacteria) |
| Hmu-DSM12286 PolB | Halomicrobium mukohataei DSM 12286 | Taxon:485914 |
| Hsa-R1 MCM | Halobacterium salinarum R-1 | Halophile, taxon:478009,strain=“R1; DSM 671” |
| Hsp-NRC1 CDC21 | Halobacterium species NRC-1 | Halophile, taxon:64091 |
| Hsp-NRC1 Pol-II | Halobacterium salinarum NRC-1 | Halophile, taxon:64091 |
| Hut MCM-2 | Halorhabdus utahensis DSM 12940 | taxon:519442 |
| Hut-DSM12940 MCM- 1 | Halorhabdus utahensis DSM 12940 | taxon:519442 |
| Hvo PolB | Haloferax volcanii DS70 | taxon:2246 |
| Hwa GyrB | Haloquadratum walsbyi DSM 16790 | Halophile, taxon:362976, strain: DSM 16790 = HBSQ001 |
| Hwa MCM-1 | Haloquadratum walsbyi DSM 16790 | Halophile, taxon:362976, strain: DSM 16790 = HBSQ001 |
| Hwa MCM-2 | Haloquadratum walsbyi DSM 16790 | Halophile, taxon:362976, strain: DSM 16790 = HBSQ001 |
| Hwa MCM-3 | Haloquadratum walsbyi DSM 16790 | Halophile, taxon:362976, strain: DSM 16790 = HBSQ001 |
| Hwa MCM-4 | Haloquadratum walsbyi DSM 16790 | Halophile, taxon:362976, strain: DSM 16790 = HBSQ001 |
| Hwa Pol-II-1 | Haloquadratum walsbyi DSM 16790 | Halophile, taxon:362976, strain: DSM 16790 = HBSQ001 |
| Hwa Pol-II-2 | Haloquadratum walsbyi DSM 16790 | Halophile, taxon:362976, strain: DSM 16790 = HBSQ001 |
| Hwa PolB-1 | Haloquadratum walsbyi DSM 16790 | Halophile, taxon:362976, strain: DSM 16790 = HBSQ001 |
| Hwa PolB-2 | Haloquadratum walsbyi DSM 16790 | Halophile, taxon:362976, strain: DSM 16790 = HBSQ001 |
| Hwa PolB-3 | Haloquadratum walsbyi DSM 16790 | Halophile, taxon:362976, strain: DSM 16790 = HBSQ001 |
| Hwa RCF | Haloquadratum walsbyi DSM 16790 | Halophile, taxon:362976, strain: DSM 16790 = HBSQ001 |
| Hwa RIR1-1 | Haloquadratum walsbyi DSM 16790 | Halophile, taxon:362976, strain: DSM 16790 = HBSQ001 |
| Hwa RIR1-2 | Haloquadratum walsbyi DSM 16790 | Halophile, taxon:362976, strain: DSM 16790 = HBSQ001 |
| Hwa Top6B | Haloquadratum walsbyi DSM 16790 | Halophile, taxon:362976, strain: DSM 16790 = HBSQ001 |
| Hwa rPol A″ | Haloquadratum walsbyi DSM 16790 | Halophile, taxon:362976, strain: DSM 16790 = HBSQ001 |
| Maeo Pol-II | Methanococcus aeolicus Nankai-3 | taxon:419665 |
| Maeo RFC | Methanococcus aeolicus Nankai-3 | taxon:419665 |
| Maeo RNR | Methanococcus aeolicus Nankai-3 | taxon:419665 |
| Maeo-N3 Helicase | Methanococcus aeolicus Nankai-3 | taxon:419665 |
| Maeo-N3 RtcB | Methanococcus aeolicus Nankai-3 | taxon:419665 |
| Maeo-N3 UDP GD | Methanococcus aeolicus Nankai-3 | taxon:419665 |
| Mein-ME PEP | Methanocaldococcus infernus ME | thermophile, Taxon:573063 |
| Mein-ME RFC | Methanocaldococcus infernus ME | Taxon:573063 |
| Memar MCM2 | Methanoculleus marisnigri JR1 | taxon:368407 |
| Memar Pol-II | Methanoculleus marisnigri JR1 | taxon:368407 |
| Mesp-FS406 PolB-1 | Methanocaldococcus sp. FS406-22 | Taxon:644281 |
| Mesp-FS406 PolB-2 | Methanocaldococcus sp. FS406-22 | Taxon:644281 |
| Mesp-FS406 PolB-3 | Methanocaldococcus sp. FS406-22 | Taxon:644281 |
| Mesp-FS406-22 LHR | Methanocaldococcus sp. FS406-22 | Taxon:644281 |
| Mfe-AG86 Pol-1 | Methanocaldococcus fervens AG86 | Taxon:573064 |
| Mfe-AG86 Pol-2 | Methanocaldococcus fervens AG86 | Taxon:573064 |
| Mhu Pol-II | Methanospirillum hungateii JF-1 | taxon 323259 |
| Mja GF-6P | Methanococcus jannaschii (Methanocaldococcus jannaschii DSM 2661) | Thermophile, DSM 2661, taxon:2190 |
| Mja Helicase | Methanococcus jannaschii (Methanocaldococcus jannaschii DSM 2661) | Thermophile, DSM 2661, taxon:2190 |
| Mja Hyp-1 | Methanococcus jannaschii (Methanocaldococcus jannaschii DSM 2661) | Thermophile, DSM 2661, taxon:2190 |
| Mja IF2 | Methanococcus jannaschii (Methanocaldococcus jannaschii DSM 2661) | Thermophile, DSM 2661, taxon:2190 |
| Mja KlbA | Methanococcus jannaschii (Methanocaldococcus jannaschii DSM 2661) | Thermophile, DSM 2661, taxon:2190 |
| Mj a PEP | Methanococcus jannaschii (Methanocaldococcus jannaschii DSM 2661) | Thermophile, DSM 2661, taxon:2190 |
| Mja Pol-1 | Methanococcus jannaschii (Methanocaldococcus jannaschii DSM 2661) | Thermophile, DSM 2661, taxon:2190 |
| Mja Pol-2 | Methanococcus jannaschii (Methanocaldococcus jannaschii DSM 2661) | Thermophile, DSM 2661, taxon:2190 |
| Mja RFC-1 | Methanococcus jannaschii (Methanocaldococcus jannaschii DSM 2661) | Thermophile, DSM 2661, taxon:2190 |
| Mja RFC-2 | Methanococcus jannaschii (Methanocaldococcus jannaschii DSM 2661) | Thermophile, DSM 2661, taxon:2190 |
| Mja RFC-3 | Methanococcus jannaschii (Methanocaldococcus jannaschii DSM 2661) | Thermophile, DSM 2661, taxon:2190 |
| Mja RNR-1 | Methanococcus jannaschii (Methanocaldococcus jannaschii DSM 2661) | Thermophile, DSM 2661, taxon:2190 |
| Mja RNR-2 | Methanococcus jannaschii (Methanocaldococcus jannaschii DSM 2661) | Thermophile, DSM 2661, taxon:2190 |
| Mja RtcB (Mja Hyp-2) | Methanococcus jannaschii (Methanocaldococcus jannaschii DSM 2661) | Thermophile, DSM 2661, taxon:2190 |
| Mj a TFIIB | Methanococcus jannaschii (Methanocaldococcus jannaschii DSM 2661) | Thermophile, DSM 2661, taxon:2190 |
| Mja UDP GD | Methanococcus jannaschii (Methanocaldococcus jannaschii DSM 2661) | Thermophile, DSM 2661, taxon:2190 |
| Mja r-Gyr | Methanococcus jannaschii (Methanocaldococcus jannaschii DSM 2661) | Thermophile, DSM 2661, taxon:2190 |
| Mja rPol A′ | Methanococcus jannaschii (Methanocaldococcus jannaschii DSM 2661) | Thermophile, DSM 2661, taxon:2190 |
| Mja rPol A″ | Methanococcus jannaschii (Methanocaldococcus jannaschii DSM 2661) | Thermophile, DSM 2661, taxon:2190 |
| Mka CDC48 | Methanopyrus kandleri AV19 | Thermophile, taxon: 190192 |
| Mka EF2 | Methanopyrus kandleri AV19 | Thermophile, taxon: 190192 |
| Mka RFC | Methanopyrus kandleri AV19 | Thermophile, taxon: 190192 |
| Mka RtcB | Methanopyrus kandleri AV19 | Thermophile, taxon: 190192 |
| Mka VatB | Methanopyrus kandleri AV19 | Thermophile, taxon: 190192 |
| Mth RIR1 | Methanothermobacter thermautotrophicus (Methanob acterium thermoautotrophicum) | Thermophile, delta H strain |
| Mvu-M7 Helicase | Methanocaldococcus vulcanius M7 | Taxon:579137 |
| Mvu-M7 Pol-1 | Methanocaldococcus vulcanius M7 | Taxon:579137 |
| Mvu-M7 Pol-2 | Methanocaldococcus vulcanius M7 | Taxon:579137 |
| Mvu-M7 Pol-3 | Methanocaldococcus vulcanius M7 | Taxon:579137 |
| Mvu-M7 UDP GD | Methanocaldococcus vulcanius M7 | Taxon:579137 |
| Neq Pol-c | Nanoarchaeum equitans Kin4-M | Thermophile, taxon:228908 |
| Neq Pol-n | Nanoarchaeum equitans Kin4-M | Thermophile, taxon:228908 |
| Nma-ATCC43099 MCM | Natrialba magadii ATCC 43099 | Taxon:547559 |
| Nma-ATCC43099 PolB-1 | Natrialba magadii ATCC 43099 | Taxon:547559 |
| Nma-ATCC43099 PolB-2 | Natrialba magadii ATCC 43099 | Taxon:547559 |
| Nph CDC21 | Natronomonas pharaonis DSM 2160 | taxon:348780 |
| Nph PolB-1 | Natronomonas pharaonis DSM 2160 | taxon:348780 |
| Nph PolB-2 | Natronomonas pharaonis DSM 2160 | taxon:348780 |
| Nph rPol A″ | Natronomonas pharaonis DSM 2160 | taxon:348780 |
| Pab CDC21-1 | Pyrococcus abyssi | Thermophile, strain Orsay, taxon:29292 |
| Pab CDC21-2 | Pyrococcus abyssi | Thermophile, strain Orsay, taxon:29292 |
| Pab IF2 | Pyrococcus abyssi | Thermophile, strain Orsay, taxon:29292 |
| Pab KlbA | Pyrococcus abyssi | Thermophile, strain Orsay, taxon:29292 |
| Pab Lon | Pyrococcus abyssi | Thermophile, strain Orsay, taxon:29292 |
| Pab Moaa | Pyrococcus abyssi | Thermophile, strain Orsay, taxon:29292 |
| Pab Pol-II | Pyrococcus abyssi | Thermophile, strain Orsay, taxon:29292 |
| Pab RFC-1 | Pyrococcus abyssi | Thermophile, strain Orsay, taxon:29292 |
| Pab RFC-2 | Pyrococcus abyssi | Thermophile, strain Orsay, taxon:29292 |
| Pab RIR1-1 | Pyrococcus abyssi | Thermophile, strain Orsay, taxon:29292 |
| Pab RIR1-2 | Pyrococcus abyssi | Thermophile, strain Orsay, taxon:29292 |
| Pab RIR1-3 | Pyrococcus abyssi | Thermophile, strain Orsay, taxon:29292 |
| Pab RtcB (Pab Hyp-2) | Pyrococcus abyssi | Thermophile, strain Orsay, taxon:29292 |
| Pab VMA | Pyrococcus abyssi | Thermophile, strain Orsay, taxon:29292 |
| Par RIR1 | Pyrobaculum arsenaticum DSM 13514 | taxon:340102 |
| Pfu CDC21 | Pyrococcus furiosus | Thermophile, taxon:186497, DSM3638 |
| Pfu IF2 | Pyrococcus furiosus | Thermophile, taxon:186497, DSM3638 |
| Pfu KlbA | Pyrococcus furiosus | Thermophile, taxon:186497, DSM3638 |
| Pfu Lon | Pyrococcus furiosus | Thermophile, taxon:186497, DSM3638 |
| Pfu RFC | Pyrococcus furiosus | Thermophile, DSM3638, taxon: 186497 |
| Pfu RIR1-1 | Pyrococcus furiosus | Thermophile, taxon:186497, DSM3638 |
| Pfu RIR1-2 | Pyrococcus furiosus | Thermophile, taxon:186497, DSM3638 |
| Pfu RtcB (Pfu Hyp-2) | Pyrococcus furiosus | Thermophile, taxon:186497, DSM3638 |
| Pfu TopA | Pyrococcus furiosus | Thermophile, taxon:186497, DSM3638 |
| Pfu VMA | Pyrococcus furiosus | Thermophile, taxon:186497, DSM3638 |
| Pho CDC21-1 | Pyrococcus horikoshii OT3 | Thermophile, taxon:53953 |
| Pho CDC21-2E | Pyrococcus horikoshii OT3 | Thermophile, taxon:53953 |
| Pho IF2 | Pyrococcus horikoshii OT3 | Thermophile, taxon:53953 |
| Pho KlbA | Pyrococcus horikoshii OT3 | Thermophile, taxon:53953 |
| Pho LHR | Pyrococcus horikoshii OT3 | Thermophile, taxon:53953 |
| Pho Lon | Pyrococcus horikoshii OT3 | Thermophile, taxon:53953 |
| Pho Pol I | Pyrococcus horikoshii OT3 | Thermophile, taxon:53953 |
| Pho Pol-II | Pyrococcus horikoshii OT3 | Thermophile, taxon:53953 |
| Pho RFC | Pyrococcus horikoshii OT3 | Thermophile, taxon:53953 |
| Pho RIR1 | Pyrococcus horikoshii OT3 | Thermophile, taxon:53953 |
| Pho RadA | Pyrococcus horikoshii OT3 | Thermophile, taxon:53953 |
| Pho RtcB (Pho Hyp-2) | Pyrococcus horikoshii OT3 | Thermophile, taxon:53953 |
| Pho VMA | Pyrococcus horikoshii OT3 | Thermophile, taxon:53953 |
| Pho r-Gyr | Pyrococcus horikoshii OT3 | Thermophile, taxon:53953 |
| Psp-GBD Pol | Pyrococcus species GB-D | Thermophile |
| Pto VMA | Picrophilus torridus, DSM 9790 | DSM 9790, taxon:263820, Thermoacidophile |
| Smar 1471 | Staphylothermus marinus F1 | taxon:399550 |
| Smar MCM2 | Staphylothermus marinus F1 | taxon:399550 |
| Tac-ATCC25905 VMA | Thermoplasma acidophilum, ATCC 25905 | Thermophile, taxon:2303 |
| Tac-DSM1728 VMA | Thermoplasma acidophilum, DSM1728 | Thermophile, taxon:2303 |
| Tag Pol-1 (Tsp-TY Pol- 1) | Thermococcus aggregans | Thermophile, taxon:110163 |
| Tag Pol-2 (Tsp-TY Pol- 2) | Thermococcus aggregans | Thermophile, taxon:110163 |
| Tag Pol-3 (Tsp-TY Pol- 3) | Thermococcus aggregans | Thermophile, taxon:110163 |
| Tba Pol-II | Thermococcus barophilus MP | taxon:391623 |
| Tfu Pol-1 | Thermococcus fumicolans | Thermophilem, taxon:46540 |
| Tfu Pol-2 | Thermococcus fumicolans | Thermophile, taxon:46540 |
| Thy Pol-1 | Thermococcus hydrothermalis | Thermophile, taxon:46539 |
| Thy Pol-2 | Thermococcus hydrothermalis | Thermophile, taxon:46539 |
| Tko CDC21-1 | Thermococcus kodakaraensis KOD1 | Thermophile, taxon:69014 |
| Tko CDC21-2 | Thermococcus kodakaraensis KOD1 | Thermophile, taxon:69014 |
| Tko Helicase | Thermococcus kodakaraensis KOD1 | Thermophile, taxon:69014 |
| Tko IF2 | Thermococcus kodakaraensis KOD1 | Thermophile, taxon:69014 |
| Tko KlbA | Thermococcus kodakaraensis KOD1 | Thermophile, taxon:69014 |
| Tko LHR | Thermococcus kodakaraensis KOD1 | Thermophile, taxon:69014 |
| Tko Pol-1 (Pko Pol-1) | Pyrococcus/ Thermococcus kodakaraensis KOD1 | Thermophile, taxon:69014 |
| Tko Pol-2 (Pko Pol-2) | Pyrococcus/Thermococcus kodakaraensis KOD1 | Thermophile, taxon:69014 |
| Tko Pol-II | Thermococcus kodakaraensis KOD1 | Thermophile, taxon:69014 |
| Tko RFC | Thermococcus kodakaraensis KOD1 | Thermophile, taxon:69014 |
| Tko RIR1-1 | Thermococcus kodakaraensis KOD1 | Thermophile, taxon:69014 |
| Tko RIR1-2 | Thermococcus kodakaraensis KOD1 | Thermophile, taxon:69014 |
| Tko RadA Tko TopA | Thermococcus kodakaraensis KOD1 Thermococcus kodakaraensis KOD1 | Thermophile, taxon:69014 Thermophile, taxon:69014 |
| Tko r-Gyr | Thermococcus kodakaraensis KOD1 | Thermophile, taxon:69014 |
| Tli Pol-1 | Thermococcus litoralis | Thermophile, taxon:2265 |
| Tli Pol-2 | Thermococcus litoralis | Thermophile, taxon:2265 |
| Tma Pol | Thermococcus marinus | taxon:187879 |
| Ton-NA1 LHR | Thermococcus onnurineus NA1 | Taxon:523850 |
| Ton-NA1 Pol | Thermococcus onnurineus NA1 | taxon:342948 |
| Tpe Pol | Thermococcus peptonophilus strain SM2 | taxon:32644 |
| Tsi-MM739 Lon | Thermococcus sibiricus MM 739 | Thermophile, Taxon:604354 |
| Tsi-MM739 Pol-1 | Thermococcus sibiricus MM 739 | Taxon:604354 |
| Tsi-MM739 Pol-2 | Thermococcus sibiricus MM 739 | Taxon:604354 |
| Tsi-MM739 RFC | Thermococcus sibiricus MM 739 | Taxon:604354 |
| Tsp AM4 RtcB | Thermococcus sp. AM4 | Taxon: 246969 |
| Tsp-AM4 LHR | Thermococcus sp. AM4 | Taxon:246969 |
| Tsp-AM4 Lon | Thermococcus sp. AM4 | Taxon:246969 |
| Tsp-AM4 RIR1 | Thermococcus sp. AM4 | Taxon:246969 |
| Tsp-GE8 Pol-1 | Thermococcus species GE8 | Thermophile, taxon: 105583 |
| Tsp-GE8 Pol-2 | Thermococcus species GE8 | Thermophile, taxon: 105583 |
| Tsp-GT Pol-1 | Thermococcus species GT | taxon:370106 |
| Tsp-GT Pol-2 | Thermococcus species GT | taxon:370106 |
| Tsp-OGL-20P Pol | Thermococcus sp. OGL-20P | taxon:277988 |
| Tthi Pol | Thermococcus thioreducens | Hyperthermophile |
| Tvo VMA | Thermoplasma volcanium GSS1 | Thermophile, taxon:50339 |
| Tzi Pol | Thermococcus zilligii | taxon:54076 |
| Unc-ERS PFL | uncultured archaeon GZfos13E1 | isolation _source=“Eel River sediment”, clone=“GZfos13E1”, taxon:285397 |
| Unc-ERS RIR1 | uncultured archaeon GZfos9C4 | isolation _source=“Eel River sediment”, taxon:285366, clone=“GZfos9C4” |
| Unc-ERS RNR | uncultured archaeon GZfos10C7 | isolation_source=“Eel River sediment”, clone=“GZfos 1 OC7”, taxon:285400 |
| Unc-MetRFS MCM2 | uncultured archaeon (Rice Cluster I) | Enriched methanogenic consortium from rice field soil,taxon: 198240 |

The split inteins of the disclosed compositions or that can be used in the disclosed methods can be modified, or mutated, inteins. A modified intein can comprise modifications to the INT_{N} segment, the INT_{C} segment, or both. The modifications can include additional amino acids fused to the N-terminus the C-terminus regions of either segment of the split intein, or can be within the either segment of the split intein. Table 3 shows a list of amino acids, their abbreviations, polarity, and charge.

TABLE 3

Amino Acids

| | | | | |
|---|---|---|---|---|
| Amino Acid | 3-Letter Code | 1-Letter Code | Polarity | Charge |
| Alanine | Ala | A | nonpolar | neutral |
| Arginine | Arg | R | Basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | Basic polar | Positive (10%) Neutral (90%) |
| Isoleucine | Ile | I | nonpolar | neutral |
| Leucine | Leu | L | nonpolar | neutral |
| Lysine | Lys | K | Basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

Once obtained, the Cognate Binding Partner and the N-Intein Ligand can be separated and purified by appropriate combinations of known techniques. These methods include, for example, methods utilizing solubility such as salt precipitation and solvent precipitation; methods utilizing the difference in molecular weight such as dialysis, ultrafiltration, gel-filtration, and SDS-polyacrylamide gel electrophoresis; methods utilizing a difference in electrical charge such as ion-exchange column chromatography; methods utilizing specific affinity such as affinity chromatography; methods utilizing a difference in hydrophobicity such as reverse-phase high performance liquid chromatography; and methods utilizing a difference in isoelectric point, such as isoelectric focusing electrophoresis. These are discussed in more detail below.

### C. Compositions and Systems for Protein Purification

Disclosed herein are protein purification systems, wherein the system comprises an intein complex complex covalently immobilized on a solid support, wherein 10, 20, 30, 40, 50, 60, 70, 80, or 90% or more of the N-Intein Ligand comprising the intein complex are associated with a Cognate Binding Partner, and wherein 10, 20, 30, 40, 50, 60, 70, 80, or 90% or more of the Cognate Binding Partners are not expressed in fusion to a desired protein of interest.

The N-Intein Ligand can be folded with a cognate binding partner to stabilize the N-Intein Ligand, as well as to increase the soluble recovery of the N-Intein Ligand, while the N-Intein Ligand is being processed and covalently immobilized on a solid support substrate. Furthermore, the N-Intein Ligand and the Cognate Binding Partner, when associated and folded within an intein complex, have a more uniform size and charge distribution than the N-Intein Ligand alone, which can mitigate downstream processing complexity.

Also disclosed is a chromatographic resin comprising a base resin with covalently-bound N-Intein Ligands, wherein the resin’s measured compressibility differential (ΔC) is less than about 1, 2, 3, 4, 5, 6, 7, 8, 9, or 10%, as compared to its base resin substrate. As defined herein, a “base resin” refers to the resin support substrate which has not had an N-Intein Ligand or any other ligand attached to it. A definition of “compressibility differential (ΔC)” is provided elsewhere herein.

Also disclosed is a chromatographic resin comprising a base resin with covalently-bound N-Intein Ligands, wherein the resin’s measured intrinsic functional compressibility factor (IFCF) is between 1.10 and 1.25. A definition of “intrinsic functional compressibility factor” (IFCF) is provided elsewhere herein.

It should be noted that the compressibility differential and intrinsic functional compressibility factors of the disclosed resin(s) are understood to be a unique mechanical property resulting from stabilization of the attached N-Intein Ligands, which is induced by the presence of a cognate binding partner. Therefore, given a particulate media comprising N-Intein Ligands covalently attached to a solid resin, a compressibility differential of ΔC < 10% and/or an intrinsic functional compressibility factor (IFCF) between 1.10 and 1.25 can indicate the presence of a cognate binding partner.

As discussed in relation to the methods above, the N-Intein Ligands covalently attached to the resin can be stabilized by Cognate Binding Partners. The Cognate Binding Partner can comprise a C-terminal intein segment (INT_{C}). The N-Intein Ligands can be stabilized via association with a Cognate Binding Partners in any processing step preceeding the ligand’s covalent immobilization to the resin substrate. The N-Intein Ligand density on the solid surface can be greater than 10 mg of N-Intein Ligand/mL resin volume. The N-Intein Ligand can be derived from a native intein, such as an Npu DnaE intein. The Cognate Binding Partner can be derived from an Npu DnaE intein. The N-Intein Ligand can comprise a purification tag and an INT_{N} segment. The N-Intein Ligand may not comprise any cysteine residues within the INTN portion of the N-Intein Ligand. The N-Intein Ligand can comprise a naturally occurring INTN segment that has been modified so that at least one internal cysteine residue has been mutated to at least one serine residue. The purification tag can comprise one or more histidine residues.

In the packed resin bed described herein, the N-Intein Ligand can comprise one or more amino acids constituting an immobilization moiety. The amino acids can be encoded to be expressed in direct fusion to or operably linked to the C-terminus of the INT_{N} segment. The one or more amino acids within the immobilization moiety can be cysteine residues. The N-Intein Ligand can further comprise a sensitivity-enhancing motif, which renders it highly sensitive to extrinsic conditions. The sensitivity-enhancing motif can be in the N-terminus region of the N-Intein Ligand. The extrinsic condition can be pH, temperature, zinc, or a combination of these. The N-Intein Ligand can comprise SEQ ID NO: 2, 3, 4, 5, 6, 7, 8, or 9. The Cognate Binding Partner can comprise SEQ ID NO: 10, 11, 12, 13, 14, 15, or 16.

Importantly, in this specific example of a protein purification system, the Cognate Binding Partner is not expressed in fusion with a protein of interest. What is meant by this is that the Cognate Binding Partner does not include, or is not linked, bound, or associated with, a protein or peptide that is desired as the end-product of the protein purification system itself during the manufacturing process. This distinguishes it from previous protein purification systems, as well as from the “secondary” use of this protein purification system, where the N-Intein Ligand associates (binds) to an INT_{C} segment expresses in fusion with a desired protein of interest. It is also important to note that the Cognate Binding Partner described herein may be expressed in fusion with other proteins or peptides, such as linker or tag moieties described previously.

Also disclosed herein is a solid affinity capture media, wherein the capture media comprises N-Intein Ligands covalently attached to its surface, further wherein less than 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, or 50%, but greater than 0.001, 0.10, 0.20, 0.30, 0.40, 0.50, 0.60, 0.70, 0.80, 0.90, 1.0, 5.0, or 10% (or any amount above, between, or below this amount) of the attached N-Intein Ligands are associated with Cognate Binding Partners (have formed an Intein Complex), and wherein 50, 60, 70, 80, 90, or 100% % (or any amount above, between, or below this amount) of the cognate binding partners are not associated with desired protein of interest.

This composition describes the properties of the affinity capture media after the intein complex has been exposed to a solid substrate, and the N-Intein Ligand has been immobilized to the substrate surface, and the Cognate Binding Partner has been dissociated from the N-Intein Ligand, and non-bound material, including the majority fraction of the Cognate Binding Partner, has been removed. It is noted that when the resin is exposed to conditions that disrupt association, and then washed, a residual amount of the N-Intein Ligand will remain associated with their Cognate Binding Partners. This creates a capture media with a unique composition which does not exist except when practicing the specific manufacturing method utilizing a cognate binding partner, as described herein.

Also disclosed herein are kits. A kit, for example, can include intein complex as described herein. Importantly, the intein complex can be made up of an N-Intein Ligand and a Cognate Binding Partner, wherein the Cognate Binding Partner does not include a desired protein of interest. The kit can comprise a vector or vectors encoding the cognate complex. For example, the kit can comprise one vector encoding the N-terminal intein, and another vector encoding the cognate binding partner. In another example, they can be encoded by the same vector. The kit can also include instructions for use.

### D. Experimental

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regards as their invention. Efforts have been made to ensure accuracy with respect to numbers (e.g., amounts, temperatures, etc.), but some errors and deviations should be accounted for.

### Example 1: SDS Page Analysis Comparing Cell Lysates of N-Intein Ligand

Expressions of N-Intein Ligand (SEQ ID No: 5) were performed under identical culture conditions in three separate 1.0 L culture batches. After each expression culture batch, cells were harvested and aliquoted to examine ligand solubility. Sample aliquots were resuspended in lysis buffer at the indicated concentrations and lysed under identical conditions.

The results can be seen in FIG. 1. Lanes are marked by type: Whole-Cell Lysate (WCL), Clarified Lysate (CL), and Pellet (P) samples. WCL lanes indicate the total cellular protein production; CL lanes represent the fraction protein that remains soluble throughout clarification of the lysate, and P lanes represent the fraction of insoluble protein that is lost when centrifuging the lysate. A crude approximation of the N-Intein Ligand’s solubility can be estimated by visually comparing the size and intensity of the Ligand band (arrow) for each batch. This is done by estimating the amount of soluble ligand appearing in lane CL as a fraction of the total ligand initially present in lane WCL for the same lysis batch.

Again, turning to FIG. 1, comparisons of expression batches A and B illustrate the characteristic batch-to-batch variability in the fraction of total ligand that remains soluble. Canonically, protein solubility is determined in vivo, primarily presumed a result of properly formed secondary and tertiary structures. However, analysis of multiple lots taken from expression batch C demonstrate that post-expression processing can have a drastic effect on the solubility of the N-Intein Ligand. For example, lysis of lot B-1 appears to show ligand solubility in excess of 90%, which would imply ‘proper’ in vivo synthesis has been achieved in expression batch B. However, when replicating lysis and centrifugation on a second aliquot from batch B one day later (lot B-2), the apparent solubility drops to <10%, despite being sourced from the same expression culture and lysed under identical conditions. Lane P from lot B-2 confirms that nearly all the ligand initially present in the lysate precipitated during centrifugation. This data shows the N-Intein Ligand is unstable and can form insoluble aggregates regardless of proper in vivo synthesis and folding.

### Example 2: Co-Expression With a Cognate Binding Partner

Conventional single-product overexpression was compared to co-expression with a Cognate Binding Partner by performing side-by-side 1.0 L expression batches under identical culture conditions. Each batch was inoculated with E. coli (BLR) strains transformed with pET vectors encoding the respective expression constructs being compared. The control batch (Conventional single-product overexpression) was transformed with a vector encoding the N-Intein Ligand alone (SEQ ID No: 5). A Co-expression batch (Co-expression of Ligand + CBP-GFP Fusion) was transformed with a bicistronic vector, separately encoding N-Intein Ligand (SEQ ID No: 5) and a Cognate Binding Partner-GFP tag fusion (SEQ ID No: 13) for concurrent co-expression. A second co-expression batch (Co-expression of Ligand + CBP) was transformed with a different bicistronic vector, separately encoding N-Intein Ligand (SEQ ID No: 5) and a Cognate Binding Partner (SEQ ID No: 14) for concurrent co-expression. All batches were processed side-by-side, 10 mL aliquots of LB growth media were inoculated from LB-agar plates and grown for ~16 hr at 37° C. using ampicillin as a selection marker. These seed cultures were then used to inoculate flasks containing 1.0 L of enriched growth media and ampicillin, then grown in a shaking incubator at 37° C. Once the cultures reached mid-log phase (OD₆₀₀ = ~5.0), expression was induced with addition of IPTG to a final concentration of 1.0 mM, and the incubator temperature was reduced to 20° C. to promote proper folding and solubility. The induced cultures were incubated while shaking for an additional ~16 hr, then separately harvested by centrifugation and weighed. The cells harvested from each batch were resuspended in lysis buffer proportional to their wet-cell weight, effectively normalizing the concentration of each batch to its culture cell density. Aliquots of each normalized resuspension were lysed mechanically, sampled, then centrifuged at 20,000 x g for 10 minutes to clarify the lysate. The clarified lysate was sampled, decanted, and the residual solids were then resuspended in an equivalent volume of buffer, then sampled again. These samples: Whole-Cell Lysate (WCL), Clarified Lysate (CL), and Pellet (P), respectively, were then analyzed via SDS-PAGE to examine ligand solubility in each expression culture.

The results shown in FIG. 2 indicate that co-expressing the Cognate Binding Partner (CBP) in vivo increases the metabolic burden on the cell. Cellular resources are finite, and introducing a secondary co-expression product therefore consumes critical materials and energy that the cell could otherwise allocate toward synthesis of the primary overexpression product.

Furthermore, the Cognate Binding Partner stabilizes a Ligand on a 1:1 stoichiometric basis, meaning the addition of a Cognate Binding Partner is structurally beneficial for the Ligand only when the Cognate Binding Partner is present in equivalent or excess molar quantities. This implies that any useful co-expression of the Cognate Binding Partner requires that it be produced in quantities proportional to the Ligand, thus consuming a significant portion of the cell’s limited resources, which effectively reduces the total production titer of the Ligand.

In FIG. 2, this effect can clearly be seen by comparing the WCL lane from each processing method: in conventional overexpression of a single Ligand product, the greater size and density of the Ligand band indicates higher levels of expression relative to the corresponding WCL lane of the Ligand co-expressed with a Cognate Binding Partner.

Because Cognate Binding Partner co-expression reduces the production titer of the Ligand, it was not expected that introducing a Cognate Binding Partner would positively impact the net productivity of the manufacturing process. Indeed, when considering also that association with the Cognate Binding Partner functionally inactivates the Ligand, requiring further processing step to strip the Cognate Binding Partner and reactivate the Ligand, this approach is actually rather counterintuitive.

However, increases in Ligand stability and solubility induced by the CBP can have positive effects elsewhere in the manufacturing process that can offset the relative reduction in Ligand product titer caused by Cognate Binding Partner co-expression.

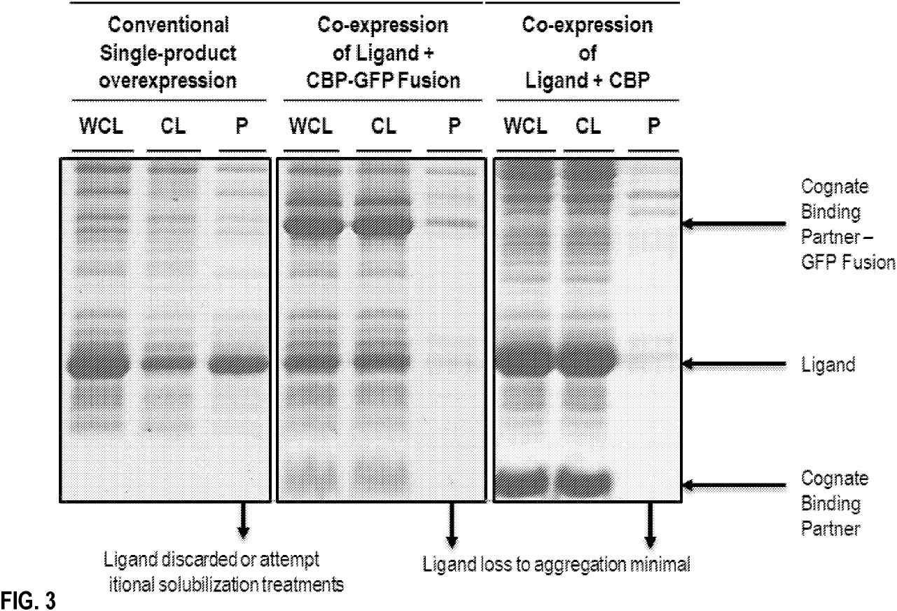

As can be seen in FIG. 3, the presence of a Cognate Binding Partner clearly has a dramatic effect on the solubility of the ligand. This effect is observed both for (SEQ ID No: 13) and (SEQ ID No: 14), despite differing mutations within their respective INT_{C}-derived domains, as well as the presence (or absence) of the GFP and His₆ tags expressed in fusion with the Cognate Binding Partner. This supports the notion that various Cognate Binding Partners could be devised to enhance the solubility of an N-Intein Ligand - so long as the critical ability to induce formation of an intein complex is preserved, mutations within the Cognate Binding Partner and/or permutations with various fusion partners can be made trivially. This trend can also be observed with several other Cognate Binding Partners - such as any of those listed from SEQ ID No: 10 through SEQ ID No: 16.

### Example 3: Ligand Solubility

FIG. 4 shows Coomassie stained SDS-PAGE analysis for each batch showing Whole-Cell Lysate (WCL), Clarified Lysate (CL), and Pellet (P) samples. WCL lanes indicate the total cellular production titer of the Ligand; P lanes show the relative fraction of Ligand that is lost when the insoluble debris is centrifuged and discarded; CL lanes represent the feedstock containing the fraction of soluble Ligand (arrows) that is available to be loaded and captured by subsequent IMAC purifications.

FIG. 4 also shows chromatograms tracing absorbance at 280 nm (A280) throughout parallel IMAC purifications performed on conventional single-product overexpression (top) and CBP co-expression (bottom) batches. A280 provides a quantitative estimate of the total protein concentration in the mobile phase as it exits the outlet of each IMAC column. The total quantity of Ligand recovered in each purification can be estimated by integrating A280 peaks occurring during the elution phase (Normalized Retention Volume > 21 CV). Samples taken from peaks labeled E1 and E2 were further analyzed by SDS-PAGE to assess purity and confirm accurate A280 quantification, as shown in the panel on the right.

FIG. 4 shows SDS-PAGE analysis of samples taken from parallel IMAC elution peaks E1 (conventional single-product overexpression) and E2 (CBP co-expression). Each fraction shows highly purified and concentrated ligand product, with similar degrees of slight contamination from co-purified host-cell proteins. The total mass of Ligand recovered by each IMAC purification was calculated by integrating the A280 signal throughout the elution phase. To account for differences in cell density between expression batches, the total mass recovered in each elution is normalized to the total biomass (wet cell weight) that is lysed to prepare the feedstock for that purification. This normalized yield is reported for each purification below its corresponding elution lane.

### Example 4: End-Use Purification and Cleaving Kinetics

Two batches of intein capture resin were manufactured with the same immobilized N-Intein Ligand (SEQ ID No: 5). The first batch was manufactured using conventional single-product overexpression and standard bioprocessing techniques, the second using the novel manufacturing process claimed herein.

For the novel manufacturing process, the N-Intein Ligand (SEQ ID No: 5) was co-expressed with a Cognate Binding Partner (SEQ ID No: 13). The co-expression products bind one another, forming an intein complex which is then purified, concentrated, buffer exchanged, and covalently immobilized on a chromatography resin. The resin was then treated with a 6 M GdnHCl gradient wash to dissociate the complex and refold the N-Intein Ligand. Since the immobilization reaction occurs selectively with the N-Intein Ligand, the Ligand is retained by its covalent bond to the resin while the dissociated Cognate Binding Partner is washed away. This “activates” the resin so that the N-Intein Ligand is now free to capture an INT_{C}-tagged protein of interest.

After manufacturing was completed, gravity-flow chromatography columns were packed with resin from each batch and used to perform identical side-by-side purifications of an INT_{C}-tagged protein of interest (SEQ ID No: 17). For these purifications, a single batch of lysate containing the INT_{C}-tagged protein of interest was processed from a single expression batch, then split equally and applied to each column to ensure comparability in assessing the performance of each resin batch. These purifications also demonstrate the intended end use of the intein capture media.

In FIG. 5, the upper panel shows the performance of the conventionally manufactured material, which appears to differ only superficially from that of the lower panel, where the capture media was manufactured using the methods disclosed herein. This comparison demonstrates that a strong chaotrope wash (6 M GdnHCl) can effectively dissociate a Cognate Binding Partner from an intein complex and reactivate the immobilized N-Intein Ligand. By extention, this also demonstrates that the presence of the Cognate Binding Partner during manufacturing does not adversely affect the performance of the final product (the intein capture media).

### Example 5: Column Packing of Chromatography Resin Aided By Cognate Binding Partner

A batch of purified N-Intein Ligand was prepared using the novel Cognate Binding Partner stabilization techniques claimed herein. As illustrated in FIG. 7, E. coli (BLR) was transformed with a single-vector bicistronic plasmid to separately encode an N-Intein Ligand (SEQ ID No: 18) and a Cognate Binding Partner (SEQ ID No: 13) for in vivo ligand stabilization. The N-Intein Ligand and Cognate Binding Partner were co-expressed, harvested, and purified using standard preparative liquid chromatography techniques. The resulting product - an Intein Complex formed by spontaneous association of the N-Intein Ligand and Cognate Binding Partner - was then aliquoted into two reaction batches for covalent immobilization onto chromatography resin.

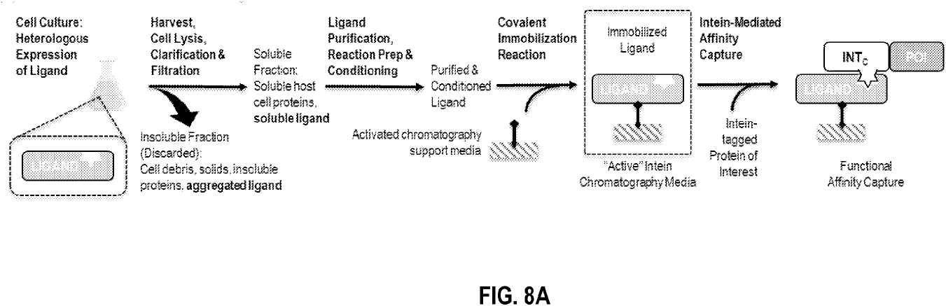

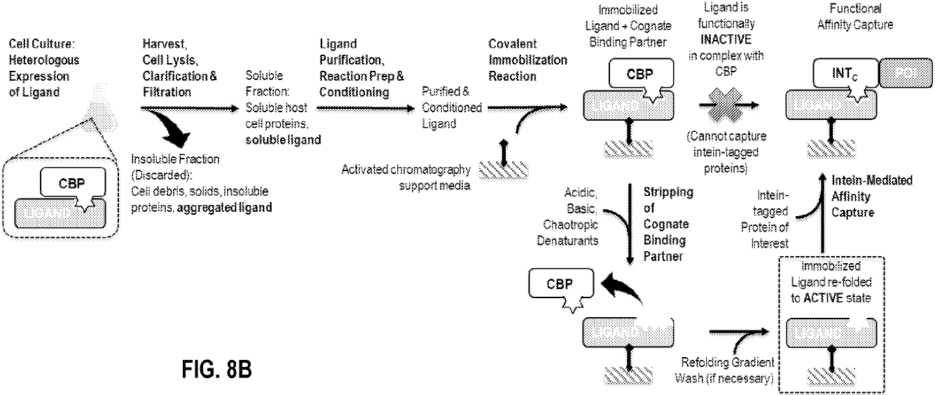

Immobilization reactions were performed using a 6% crosslinked agarose chromatography resin (mean particle size dₚ = 90 µm) which was derivatized with thiol-reactive functional groups. The purification aliquots were reacted with this resin to selectively conjugate the N-Intein Ligand via its engineered Cysteine immobilization moiety. Each reaction batch was then passivated with excess thiol to inactivate any remaining immobilization sites on the resin. Following reaction and passivation, the first resin reaction batch (denoted “- CBP”) was subjected to a denaturing low-pH stripping treatment in a stirred vessel to dissociate and remove the Cognate Binding Partner from the resin (as illustrated in FIGS. 7 and 8(b)). The second resin reaction batch (denoted “+ CBP”) was left untreated, allowing the Cognate Binding Partner to remain complexed to the resin-immobilized N-Intein Ligand. This enables direct comparison and evaluation of resin properties when the N-Intein Ligand is stabilized by a Cognate Binding Partner. Both batches were then treated with a final wash passing >20 volume equivalents of phosphate-buffered saline (PBS) pH 7.4 through each batch to remove residual solvents, reactants, unreacted ligand, and/or dissociated Cognate Binding Partner. The resins were drained in a filter funnel, then resuspended with addition of fresh PBS, transferred to a graduated cylinder, gravity-settled for at least 12 hours, then adjusted to a 50% slurry by pipette.

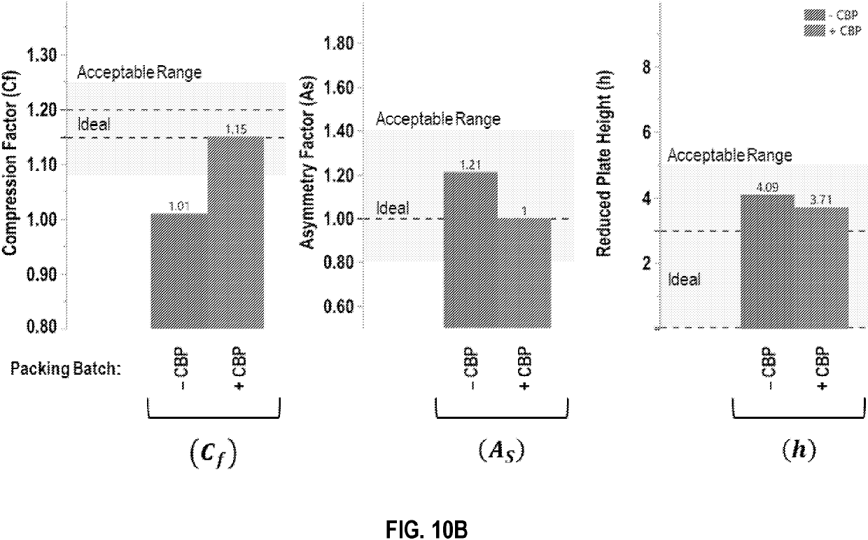

These resins were then flow-packed into identical chromatography columns side-by-side to evaluate the Cognate Binding Partner’s influence on column packing and flow uniformity throughout the packed bed. For each resin batch, 4.0 mL of 50% slurry were added to 6.6 mm diameter chromatography columns, and the remaining headspace in each column was filled with additional PBS to displace any air in the columns. The columns were then sealed with adjustable-height flow adapters at the column inlets and then connected to an FPLC. Flow adapters were initially set at an expanded position with the inlet frit ~5 cm above the settled resin bed, then PBS was pumped through the columns at a linear superficial velocity of 50 cm/hr to ensure resin settling. The heights of the settled resin beds (L₀) were measured and recorded for each column. The column inlet was then vented, and the flow adapter height was adjusted to position the inlet frit at 0.5 cm above the settled resin bed. The column inlet was then reconnected to the FPLC to begin constant-pressure flow packing: additional PBS through the column at a PID-controlled flow rate set to maintain a pressure drop across the column of ΔP = 2.0 bar. Packing flow was maintained for at least 5 minutes after bed compression stabilized, then the flow adapter was adjusted downward further until the inlet frit physically contacted the top of the compressed resin bed. FPLC flow was restarted at a constant flow rate corresponding to 50 cm/hr and pumped for an additional 5 minutes. The resin bed was visually ispected to confirm that no additional bed compression or void formation occurred duing the final packing step. The heights of the compressed resin beds (L) were measured and recorded for each column. These measurements were used to calculate the packed bed volume compression factor (C_{f}) for each resin using the formula C_{f} = L₀/L. The results are presented in FIG. 10.

After column packing was completed, a standard column efficiency test using an inert tracer pulse injection was performed for each column to evaluate flow uniformity throughout the packed beds. Each test was performed using a PBS running buffer pumped at a constant linear velocity of 50 cm/hr. After equilibration, columns were injected with a 200 µL pulse of tracer solution (PBS pH 7.4 + 1.0 M NaCl + 0.1% (v/v) acetone). Isocratic elution of the tracer was continuously monitored for an additional 5 CV by inline UV-spectroscopy; the concentration of tracer in the column effluent was indicated by absorbance at a wavelength of λ=280 nm (A₂₈₀). A chromatogram from a tracer pulse experiment performed on each resin is presented in FIG. 10(a). Applying the methodology commonly practiced by those skilled in the art illustrated in FIG. 9, these data were then used to calculate the peak asymmetry factor (Aₛ) and reduced plate height (h) for each batch to validate the quality of column packing for each resin batch. C_{f}, Aₛ and h are reported for each batch in FIG. 10(b) to demonstrate the effects of packing an intein capture resin with and without the aid of a Cognate Binding Partner.

Interestingly, the agarose resin base matrix (i.e. the base resin with no ligand immobilized) can be packed to a compression factor of C_{f} = 1.15, but once the N-Intein Ligand was conjugated (- CBP batch), the resin was no longer compressible when slurry-packed at ΔP = 2.0 bar, achieving a compression factor of only C_{f} = 1.01. Efforts to further compress the resin bed with mechanical compression resulted in asymmetry and reduced plate height test metrics outside of acceptable limits, indicating that the excess pressure was likely cracking or crushing the resin substrate, thus damaging the integrity of the packed bed. However, when packing the resin batch stabilized by a Cognate Binding Partner (+ CBP batch) under otherwise identical conditions, the compressibility of the resin is restored. As can be observed in FIG. 10(b), the + CBP was able to be slurry packed to a compression factor of C_{f} = 1.15 while maintaining acceptable asymmetry and reduced plate height test metrics, mirroring the performance of the unmodified base resin.

### WORKS CITED

Andres, A., K. Broeckhoven and G. Desmet (2015). “Methods for the experimental characterization and analysis of the efficiency and speed of chromatographic columns: A step-by-step tutorial.” Anal Chim Acta 894: 20-34.

Aranko, A. S., A. Wlodawer and H. Iwai (2014). “Nature’s recipe for splitting inteins.” Protein Engineering Design & Selection 27(8): 263-271.

Carrió, M. M. and A. Villaverde (2002). “Construction and deconstruction of bacterial inclusion bodies.” Journal of Biotechnology 96(1): 3-12.

Dyson, H. J. and P. E. Wright (2005). “Intrinsically unstructured proteins and their functions.” Nat Rev Mol Cell Biol 6(3): 197-208.

Eryilmaz, E., N. H. Shah, T. W. Muir and D. Cowburn (2014). “Structural and Dynamical Features of Inteins and Implications on Protein Splicing.” Journal of Biological Chemistry 289(21): 14506-14511.

GE-Healthcare (2010). Column efficiency testing. Application note 28-9372-07 AA.

Kastritis, P. L. and A. M. J. J. Bonvin (2013). “On the binding affinity of macromolecular interactions: daring to ask why proteins interact.” Journal of The Royal Society Interface 10(79): 20120835.

Nichols, N. M., J. S. Benner, D. D. Martin and T. C. Evans Jr (2003). “Zinc Ion Effects on Individual Ssp DnaE Intein Splicing Steps: Regulating Pathway Progression.” Biochemistry 42(18): 5301.

O’Brien, E. P., R. I. Dima, B. Brooks and D. Thirumalai (2007). “Interactions between hydrophobic and ionic solutes in aqueous guanidinium chloride and urea solutions: lessons for protein denaturation mechanism.” J Am Chem Soc 129(23): 7346-7353.

Perler, F. B. (1999). “InBase, the New England Biolabs Intein Database.” Nucleic Acids Research 27(1): 346-347.

Perler, F. B. (2002). “InBase: the Intein Database.” Nucleic Acids Research 30(1): 383-384.

Perler, F. B., E. O. Davis, G. E. Dean, F. S. Gimble, W. E. Jack, N. Neff, C. J. Noren, J. Thorner and M. Belfort (1994). “Protein splicing elements - inteins and exteins - a definition of terms and recommended nomenclature.” Nucleic Acids Research 22(7): 1125-1127.

Pontius, B. W. (1993). “Close encounters: why unstructured, polymeric domains can increase rates of specific macromolecular association.” Trends in Biochemical Sciences 18(5): 181-186.

Rathore, A. S., R. M. Kennedy, J. K. O’Donnell, I. Bemberis and O. Kaltenbrunner (2003). “Qualification of a chromatographic column: Why and how to do it.” Biopharm international 16(3): 30-40.

Rosano, G. L. and E. A. Ceccarelli (2014). “Recombinant protein expression in Escherichia coli: advances and challenges.” Front Microbiol 5: 172.

Saleh, L. and F. B. Perler (2006). “Protein splicing in cis and in trans.” Chemical Record 6(4): 183-193.

Shah, N. H., G. P. Dann, M. Vila-Perello, Z. Liu and T. W. Muir (2012). “Ultrafast protein splicing is common among cyanobacterial split inteins: implications for protein engineering.” J Am Chem Soc 134(28): 11338-11341.

Shah, N. H., E. Eryilmaz, D. Cowburn and T. W. Muir (2013). “Naturally Split Inteins Assemble through a “Capture and Collapse” Mechanism.” Journal of the American Chemical Society 135(49): 18673-18681.

Shi, J. X. and T. W. Muir (2005). “Development of a tandem protein trans-splicing system based on native and engineered split inteins.” Journal of the American Chemical Society 127(17): 6198-6206.

Shoemaker, B. A., J. J. Portman and P. G. Wolynes (2000). “Speeding molecular recognition by using the folding funnel: the fly-casting mechanism.” Proc Natl Acad Sci U S A 97(16): 8868-8873.

Southworth, M. W., E. Adam, D. Panne, R. Byer, R. Kautz and F. B. Perler (1998). “Control of protein splicing by intein fragment reassembly.” EMBO J 17(4): 918-926.

Stickel, J. J. and A. Fotopoulos (2001). “Pressure - Flow Relationships for Packed Beds of Compressible Chromatography Media at Laboratory and Production Scal.” Biotechnology Progress 17(4): 744-751.

Weber, K. and D. J. Kuter (1971). “Reversible denaturation of enzymes by sodium dodecyl sulfate.” Journal of Biological Chemistry 246(14): 4504-4509.

Wright, P. E. and H. J. Dyson (2009). “Linking folding and binding.” Curr Opin Struct Biol 19(1): 31-38.

Zettler, J., V. Schütz and H. D. Mootz (2009). “The naturally split Npu DnaE intein exhibits an extraordinarily high rate in the protein trans-splicing reaction.” FEBS Letters 583(5): 909-914.

Zheng, Y., Q. Wu, C. Wang, M.-q. Xu and Y. Liu (2012). “Mutual synergistic protein folding in split intein.” Bioscience reports 32(5): 433-442.

1. A method of stabilizing an N-Intein Ligand during expression and purification, the method comprising:
a. forming an intein complex via assembly of an N-Intein Ligand and a Cognate Binding Partner;
b. purifying the intein complex; and
c. immobilizing the intein complex to a solid support.

2. The method of claim 1, further comprising the steps of:
d. subjecting the intein complex to conditions that disrupt association between the N-Intein Ligand and the Cognate Binding Partner; and
e. providing conditions that allow the N-Intein Ligand to fold into an active state while remaining immobilized.

3. The method of claim 1, wherein the Cognate Binding Partner comprises a C-terminal intein segment.

4. The method of claim 1, wherein, in step a), the N-Intein Ligand and the Cognate Binding Partner are co-expressed in vivo.

5. The method of claim 4, wherein the N-Intein Ligand and the Cognate Binding Partner are expressed in a single cell from a single plasmid or two-plasmid system.

6. The method of claim 1, wherein, in step a), the N-Intein Ligand is exposed to the Cognate Binding Partner in trans, after expression of the N-Intein Ligand.

7. The method of claim 1, wherein, in step c), the N-terminal intein segment is covalently immobilized to the solid support.

8. The method of claim 1, wherein the solid support is a conventional chromatographic media, including a porous resin, a membrane, a monolith or a magnetic bead.

9. The method of claim 8, wherein the chromatographic media is a solid chromatographic resin backbone.

10. The method of claim 7, wherein N-Intein Ligand density on a solid support is greater than 10 mg of N-Intein Ligand/mL resin volume.

11. The method of claim 1, wherein a chaotropic agent or a basic or acidic solution can be used to create conditions that disrupt association between the N-Intein Ligand and the Cognate Binding Partner.

12. The method of claim 2, wherein disrupting association between the N-Intein Ligand and the Cognate Binding Partner is followed by a condition that causes the N-Intein Ligand to revert to an active state wherein the N-Intein Ligand can accept a new binding partner.

13. The method of claim 12, wherein the disrupting conditions include one of the following: a chaotropic agent such as guanidine hydrochloride, an acid such as phosphoric acid, or a base such as sodium hydroxide.

14. The method of claim 1, wherein the N-Intein Ligand has been derived from a native intein.

15. The method of claim 14, wherein N-Intein Ligand is derived from an Npu DnaE intein.

16. The method of claim 14, wherein the Cognate Binding Partner is derived from an Npu DnaE intein.

17. The method of claim 1, wherein the N-Intein Ligand comprises a purification tag and an INT_{N} segment.

18. The method of claim 17, wherein the N-Intein Ligand does not comprise any cysteine residues within the INT_{N} portion of the N-Intein Ligand.

19. The method of claim 17, wherein an N-Intein Ligand comprising a naturally occurring INT_{N} segment has been modified so that at least one internal cysteine residue has been mutated to at least one serine residue.

20. The method of claim 17, wherein the purification tag comprises one or more histidine residues.

21. The method of claim 1, wherein the N-Intein Ligand comprises one or more amino acids constituting an immobilization moiety.

22. The method of claim 21, wherein the amino acids are encoded to be expressed in direct fusion to or operably linked to the C-terminus of the INT_{N} segment, thereby allowing for covalent immobilization of the N-Intein Ligand.

23. The method of claim 21, wherein the one or more amino acids within the immobilization moiety are cysteine residues.

24. The method of claim 1, wherein the N-Intein Ligand further comprises a sensitivity-enhancing motif, which renders it highly sensitive to extrinsic conditions.

25. The method of claim 24, wherein the sensitivity-enhancing motif is in the N-terminus region of the N-Intein Ligand.

26. The method of claim 24, wherein the extrinsic condition is pH, temperature, zinc, or a combination of these.

27. The method of claim 1, wherein the N-Intein Ligand comprises SEQ ID NO: 2, 3, 4, 5, 6, 7, 8, 9, or 18.

28. The method of claim 1, wherein the Cognate Binding Partner comprises SEQ ID NO: 10, 11, 12, 13, 14, 15, or 16.

29-56. (canceled)
